SEQUENCE LISTING (2 sequences)

SEQ ID NO: 1 - DNA, 28 bp, Artificial Sequence
  Primer
ccccgcccggcgtgcgagccggtgtatg

SEQ ID NO: 2 - DNA, 18 bp, Artificial Sequence
  Primer
cgggcttgcggacattgg

(19) United States
(12) Patent Application Publication
(10) Pub. No.: US 2011/0142789 A1
(43) Pub. Date: Jun. 16, 2011

(54) Compositions and Methods for the Diagnosis and Treatment of Amyotrophic Lateral Sclerosis

(75) Inventors: Aaron D. Gitler, Philadelphia, PA (US); Nancy Bonini, Narberth, PA (US)

(73) Assignee: The Trustees of the University of Pennsylvania, Philadelphia, PA (US)

(21) Appl. No.: 12/965,618

(22) Filed: Dec. 10, 2010

Related U.S. Application Data
(60) Provisional application No. 61/285,308, filed on Dec. 10, 2009.

(51) Int. Cl. A61K 31/79; C12Q 1/68; C12Q 1/02; A61P 25/00; C08F 8/32

(52) U.S. Cl. 424/78.24; 435/6.1; 435/6.12; 435/29; 525/54.1

(57) ABSTRACT

Compositions and methods for diagnosis and treatment of ALS are provided.

This application claims priority to U.S. provisional Application, 61/285,308 filed Dec. 10, 2009, the entire contents of which are incorporated by reference herein.

Pursuant to 35 U.S.C. §202(c) it is acknowledged that the U.S. Government has rights in the invention described, which was made in part with funds from the National Institutes of Health, Grant Number DP2OD004417-01.

### FIELD OF THE INVENTION

This invention relates to the fields of diagnostic assays and motor neuron disease. More specifically, compositions and methods are provided which facilitate diagnosis of amyotrophic lateral sclerosis (ALS). Also provided is a screening method for identifying therapeutic agents useful for the treatment of this devastating neurodegenerative disorder.

### BACKGROUND OF THE INVENTION

Several publications and patent documents are cited throughout the specification in order to describe the state of the art to which this invention pertains. Each of these citations is incorporated herein by reference as though set forth in full.

ALS, also known as Lou Gehrig's disease, is a devastating adult onset neurodegenerative disease with no cure¹. In fact, we still know little about the causes of ALS. The disease is mostly sporadic (SALS) but approximately 10% of cases have a first or second-degree relative with ALS (familial ALS (FALS)). Mutations in SOD1, encoding Cu/Zn superoxide dismutase, have been identified in ˜20% of FALS cases², for an overall incidence of ˜2%. Additional ALS disease genes have also been identified that are even more rare. Identifying new and potentially common genetic risk factors for ALS will accelerate understanding of the disease, aid the development of biomarkers, and spur innovative new treatments.

Recently, the 43 kDa TAR DNA binding protein (TDP-43) was identified as a major player in sporadic and familial ALS. TDP-43 is a highly conserved, ubiquitously expressed protein, initially identified by virtue of its ability to bind the HIV-1 TAR DNA element and act as a transcriptional repressor³. In addition to a glycine-rich C-terminal region, TDP-43 contains two RNA recognition motifs (RRM1 and RRM2) and is able to bind UG-repeats in RNA^{4,5}.

Some reports suggest TDP-43 might play a role in regulating splicing, as a bridge for nuclear bodies via an interaction with the survival motor neuron (SMN) protein, or in microRNA biogenesis⁶. In 2006, TDP-43 was identified as the major disease protein in ubiquitinated cytoplasmic inclusions in neurons of patients with ALS and frontotemporal lobar degeneration with ubiquitinated inclusions (FTLD-TDP)⁷. Subsequently, mutations in the gene encoding TDP-43 (TARDBP) were found associated with familial cases of ALS and FTLD-TDP^{8,9}, arguing strongly for a central role of TDP-43 in disease pathogenesis. TDP-43 is normally a nuclear protein but pathological inclusions contain cytoplasmic TDP-43 aggregates, suggesting that altered subcellular localization of the protein may be critical to disease pathogenesis^{10,11} Little is known about how loss of one or more of the biological functions of TDP-43, or how a potential toxic gain-of-function, might contribute to neurodegenerative disease. Moreover, nothing is known about genetic modifiers of TDP-43 pathogenesis or how other factors that interact with TDP-43 contribute to the risk of developing ALS or the age of disease onset.

To address these deficits, we have been investigating TDP-43 pathogenesis in yeast and fly. Such simple genetic systems are powerful tools for studying human diseases including complicated disorders like neurodegeneration¹²⁻¹⁴. For example, expression of human neurodegenerative disease proteins, such as the Parkinson's disease protein a-synuclein or the Huntington's disease protein, in yeast results in aggregation and toxicity^{15,16}. We and others have used such models to define disease mechanisms and discover genetic and small molecule modifiers of pathogenesis. Importantly, several of the modifier genes discovered in yeast are efficacious in animal models of disease¹⁷⁻²¹. To define TDP-43 pathobiology²² and to determine the effects of ALS-linked TARDBP mutations in vitro and in vivo²³, we generated yeast strains with constitutive or inducible expression of human TDP-43. In yeast, TDP-43 is initially localized to the nucleus but eventually forms cytoplasmic inclusions. This is similar to the pathobiology of TDP-43 in human neurons, where TDP-43 normally exists as a nuclear protein but in disease converts to a cytoplasmic aggregate-like localization. Importantly, expressing TDP-43 is highly toxic to yeast cells, thus modeling the degenerative component of the human situation.

In an unbiased screen to define modifiers of TDP-43 toxicity in yeast, we identified Ataxin-2 as a potent, dose-sensitive modulator of TDP-43 toxicity across multiple model systems. These data implicate Ataxin-2 in TDP-43 pathobiology.

### SUMMARY OF THE INVENTION

In accordance with the present invention, a method for predicting an increased risk of an individual for developing amyotrophic lateral sclerosis (ALS) is provided. An exemplary method entails obtaining a nucleic acid sample encoding ataxin-2 from an individual and determining whether or not ataxin-2 comprises intermediate length polyglutamine expansions relative to wild-type ataxin-2 encoding nucleic acids, wherein the presence of an ataxin-2 intermediate-length polyglutamine expansion within said ataxin-2 relative to wild type ataxin-2 is indicative of an increased risk of ALS. In another aspect the method can be used to predict in increased risk of early onset ALS. In particularly preferred embodiment, the polyglutamine expansion contains between 24-34 glutamines, more preferably, the expansion contains 24-33 glutamines. Alternatively, the expansions may range between 25 to 28 glutamines.

Another embodiment of the invention comprises a diagnostic kit for performing the aforementioned method. An exemplary kit comprises reagents suitable for isolation of nucleic acids (e.g., genomic DNA) and reagents suitable for detection of ataxin-2 encoding nucleic acid comprising said CAG (glutamine) repeats. In one aspect, the kit comprises SEQ ID NO: 1 and SEQ ID NO: 2, reagents suitable for isolation of DNA from said individual, reagents suitable for performing PCR and wild type ataxin-2 and ALS associated ataxin-2 encoding nucleic acids for use as negative and positive controls.

A method for identifying agents which inhibit TDP-43-ataxin-2 complex formation is also encompassed by the present invention. One such method entails providing a cell which expresses TDP-43 and ataxin-2 containing intermediate length polyQ expansions, the expression being associated with increased cellular toxicity and cytoplasmic aggregate formation and contacting these cells with an effective amount of an agent. Cellular toxicity and/or aggregate formation is then measured in the presence of said agent relative to a non-treated control cell, wherein a decrease in cellular toxicity identifies an agent which reduces TDP-43-ataxin-2 mediated cellular toxicity and cytoplasmic aggregate formation. Agent obtained from the libraries disclosed in Example 3 may be screened in accordance with the present invention. Agents so identified, also form an aspect of the invention. Such agents, present in pharmaceutically acceptable preparations should have efficacy for the treatment and prevention of ALS.

### BRIEF DESCRIPTION OF THE DRAWINGS

FIG. 1. Ataxin-2 is a dose-sensitive modifier of TDP-43 toxicity in yeast and flies. a) Spotting assays with yeast TDP-43 showing toxicity. Five-fold serial dilutions of yeast cells were spotted onto glucose (expression repressed) or galactose (expression induced). Upregulation of PBP1, the yeast Ataxin-2 homolog, enhances TDP-43 toxicity. Whereas PBP1 has no effect on yeast viability when expressed with the control protein YFP, when co-expressed with TDP-43, it enhances the toxic effect on yeast growth. Enhancement is specific because PBP1 does not affect the toxicity of pathogenic Huntington fragment (htt72Q). b) Spotting assays with yeast TDP43 showing that deletion of the PBP1 gene (pbp1Δ) suppresses TDP-43 toxicity. Whereas expression of TDP-43 from a plasmid in WT yeast was highly toxic to growth, this toxicity was mitigated in pbp1Δ cells. The effect was specific because toxicity of the human disease protein α-synuclein (asyn) was not suppressed by pbp1Δ. c) TDP-43 causes disruption of the fly eye. Whereas a control protein (YFP) has no effect on eye structure, expression of TDP-43-YFP causes a disrupted structure associated with progressive degeneration. Experiment performed at 29° C. Genotypes: gmr-GAL4 in trans to UAS-YFP or UAS-TDP-43-YFP. d) Expression of TDP-43 causes progressive loss of motility of flies when expressed in the nervous system. Genotypes: elav-GAL4 in trans to + or UAS-TDP-43. e) Upregulation of dAtx2 enhances the toxicity of TDP-43 in Drosophila. Flies expressing TDP-43 or dAtx2 alone have a mild effect on retinal structure, which is markedly more severe when TDP-43 is co-expressed with dAtx2. Genotypes: gmr-GAL4 in trans to UAS-YFP, UAS-TDP-43, UAS-dAtx2, and UAS-TDP-43 UAS-dAtx2. f) Reduction of the level of endogenous dAtx2 mitigates TDP-43 toxicity. Whereas TDP-43 on its own causes retinal disruption, reducing dAtx2 levels by 50% improves the structure of the eye. Genotypes: UAS-TDP-43-YFP/+; gmr-GAL4/+ and UAS-TDP-43-YFP/+; gmr-GAL4/dAtx2^{X1}. g) TDP-43 causes progressive, age-dependent degeneration. Control flies at 20 d show the typical highly regular external and internal retinal structure. Flies expressing TDP-43 show mild degeneration at 1 d, which progresses to patchy loss of pigmentation on the external eye, and massive loss of the retinal tissue internally. Genotypes: control gmr-GAL4/UAS-YFP. gmr-GAL4/UAS-TDP43-YFP. h) ALS-linked mutant TDP-43 causes more severe degeneration than WT TDP-43 in Drosophila. h (i) WT TDP-43 and the ALS-linked point mutant Q331K cause disruption of the fly eye. Whereas a control protein (YFP) has no effect on eye structure, expression of TDP-43-YFP or TDP-43.Q331K-YFP cause a disrupted structure associated with progressive degeneration. The Q331K mutant causes more severe degeneration, although it is expressed at the same level as the wild-type protein (data not shown). YFP and WT TDP-43 eyes are as in FIG. 1c. Experiment performed at 29° C. Genotypes: gmr-GAL4 in trans to UAS-YFP, GAS-TDP-43-YFP or GAS-TDP-43.Q331K-YFP. h(ii)) Motility deficits upon selective expression in motor neurons. TDP-43.Q331K causes a more severe loss of motility in flies than WT TDP-43 when expressed in motor neurons of the nervous system. Genotypes: D42-GAIL in trans to +, UASTDP-3 or UAS-TDP-43.Q331K. i) TDP-43 toxicity is not modulated by Hsp70 in Drosophila. External eyes and internal retinal sections of flies expressing (left) TDP-43 alone, and (right) TDP-43 together with Hsp70. Added expression of Hsp70 has no effect on the toxicity of TDP-43. Genotypes: (left) UAS-TDP-43/+; gmr-GAL4/+ and (right) UAS-TDP-43/UAS-human Hsp70; gmr-GAL4/+.

FIG. 2. Ataxin-2 and TDP-43 physically and genetically interact in a manner dependent on RNA binding. a) Yeast cells co-expressing CFP-tagged Pbp1 and YFP-tagged TDP-43. CFP-Pbp1 and TDP-43-YFP show accumulations in yeast that frequently co-localize (arrows). b) Co-immunoprecipitation assays in yeast. Yeast cells co-transformed with untagged TDP-43 and either CFP-Pbp1 or CFP alone were lysed and subjected to immunoprecipitation with an α-GFP antibody (also detects CFP), then subjected to immunoblotting with a-TDP43. CFP-Pbp1 immunoprecipitated with TDP-43, but CFP did not. c) TDP-43 and Ataxin-2 physically interact in mammalian cells in a manner dependent on the RRM motifs. HEK293T cells were transfected with expression constructs encoding YFP, TDP-43-YFP, TDP-43_{ΔNLS}-YFP (NLS mutant that localizes the protein to the cytoplasm), TDP-43_{ΔNLS,5F}_{_{→}}_{L}-YFP (NLS mutant coupled with RNA-binding mutant, (5 point mutations in the RRM domains that abolish RNA-binding)), or TDP-43_{5F}_{_{→}}_{L}-YFP (RNA-binding mutant). Protein was immunoprecipitated with a-GFP antibody (detects YFP), and then subjected to immunoblotting with a-Ataxin-2 to detect endogenous Ataxin-2. Whereas TDP-43 and TDP-43_{ΔNLS} both interact with Ataxin-2, the RNA-binding mutant versions do not. d) Co-IP in HEK293T cells as in (c), but now with lysates treated with RNase. The interaction between Ataxin-2 and TDP-43 seen normally (left lanes) was abolished upon RNase treatment (right lanes). e) HEK293T cells transfected with YFP-tagged WT and mutant TDP-43 constructs then immunostained for endogenous Ataxin-2. Normally, Ataxin-2 is localized to the cytoplasm forming occasional cytoplasmic accumulations. TDP-43 localized to the nucleus in a diffuse pattern. TDP-43_{ΔNLS} localized to the cytoplasm where it occasionally formed cytoplasmic aggregates; these aggregates always co-localized with Ataxin-2 cytoplasmic accumulations (arrow). Abolishing the ability of TDP-43 ability to interact with RNA with TDP-43_{ΔNLS,5}_{_{→}}_{L}, or TDP-43₅_{_{→}}_{L}, (not shown) eliminated Ataxin-2 colocalization (arrowheads). f) Spotting assays with yeast for TDP-43 toxicity. Whereas WT and TDP-43_{ΔNLS} constructs are toxic, mutations of TDP-43 that prevent RNA binding (TDP-43_{ΔNLS,5}_{_{→}}_{L}, and TDP-43_{5F}_{_{→}}_{L}) abolish TDP-43 toxicity.

FIG. 3. Ataxin-2 localization is perturbed in ALS patient neurons, and TDP-43 is mislocalized to the cytoplasm and aggregated in SCA2 patient neurons. a-d, immunostaining for Ataxin-2 in spinal cord neurons. a) In control spinal cord neurons, Ataxin-2 is localized throughout the cytoplasm in a diffuse granular pattern. (b-f) In ALS patient spinal cord neurons, Ataxin-2 was present in distinct cytoplasmic accumulations (arrows). Ataxin-2 immunostaining in ALS patient spinal cord neurons (b-d) resembled. TDP-43 pre-inclusions (h,i below). In some cases, Ataxin-2-positive accumulations were adjacent to clearings indicative of TDP-43 aggregates (* in (b)). e, Quantitation of Ataxin-2 large accumulations in control (neurologically normal) vs. ALS patient spinal cord neurons from a blinded analysis. 27.2+/−12.3% of spinal cord neurons in ALS patients had large accumulations of Ataxin-2 compared to 4.7+/−2.6% of control spinal cord neurons. g-j, TDP-43 immunostaining of control and ALS patient spinal cord neurons. g) Normal nuclear localization. h-j) TDP-43 immunostaining of ALS patient spinal cord neurons, illustrating TDP-43 positive pre-inclusions (h,i arrows), and j) larger round (arrow) and skein-like aggregates (arrowheads). k-o), TDP-43 immunostaining of SCA2 and control tissue. k) Control cerebellum showing normal nuclear TDP-43 staining in Purkinje cells (arrows). l) SCA2 cerebellar Purkinje neurons. TDP-43 immunoreactivity is concentrated in the nucleus, but also extrudes into the dendritic arbor, with some dendritic branches displaying the corkscrew shape typical of neurodegenerative processes (arrowheads). m) SCA2 brainstem motor neuron in the hypoglossus nucleus. TDP-43 aggregates protrude from the nucleus into the cytoplasm (arrow). n) SCA2 brainstem abducens nucleus. A TDP-43 aggregate (arrow) remains in the neuropil between motor neurons. o) SCA2 brainstem locus coeruleus neuron. Thread-like inclusion along the nuclear membrane and throughout a neurite (arrows).

FIG. 4. Intermediate-length Ataxin-2 polyQ expansions linked to ALS. a) The ATXN2 gene contains a trinucleotide repeat encoding polyQ. The polyQ repeat length is normally 22-23. Expansions of >34 cause SCA2²⁷. We hypothesized that intermediate-length polyQ expansions (˜24-34) could be linked to ALS. The Ataxin-2 polyQ length was defined by Genescan analysis of ALS cases and neurologically normal controls (for details, see Table 1 and Methods). b) Representative examples of Genescan analysis of polyQ lengths from control and ALS cases. The size of each allele is indicated. (c-d) To determine the effect of intermediate-length polyQ expansions on Ataxin-2, Ataxin-2 protein stability and steady state levels were determined from control (n=4, all with Ataxin-2 polyQ lengths of 22) and ALS patient-derived cells with intermediate-length polyQ expansions (n=4, Ataxin-2 polyQ lengths 24, 27, 29, and 31) lymphoblastoid cell lines. c) Although steady-state levels of Ataxin-2 were comparable between control and intermediate-length polyQ repeat cells, cycloheximide treatment revealed an increase in stability of Ataxin-2 with intermediate-length repeat expansions compared to Ataxin-2 with normal polyQ length (d). We did not detect a difference in Ataxin-2 stability between control and ALS patient-derived cells from non-expanded cases.

### DETAILED DESCRIPTION OF THE INVENTION

Amyotrophic lateral sclerosis (ALS) is a devastating human neurodegenerative disease. The causes of ALS are poorly understood, although the protein. TDP-43 has been suggested to play a critical role in disease pathogenesis. Here we show that Ataxin-2, a polyglutamine (polyQ) protein mutated in spinocerebellar ataxia type 2 (SCA2), is a potent modifier of TDP-43 toxicity in animal and cellular models. The proteins associate in a complex that depends on RNA. Ataxin-2 is abnormally localized in spinal cord neurons of ALS patients. Likewise, TDP-43 shows mislocalization in SCA2. To assess a role in ALS, we analyzed the Ataxin-2 gene (ATXN2) in >600 ALS patients. We found intermediate-length polyQ expansions (24-33 Qs) in ATXN2 significantly associated with ALS (4.5% of cases, P=2.1×10⁻⁴). Moreover, Ataxin-2 intermediate-length repeats were associated with a >10 year advanced age of disease onset. These data establish ATXN2 as a new and relatively common ALS disease gene.

### DEFINITIONS

“Amyotrophic lateral sclerosis (ALS)” is a progressive neurodegenerative disease that affects nerve cells in the brain and the spinal cord. Motor neurons reach from the brain to the spinal cord and from the spinal cord to the muscles throughout the body. The progressive degeneration of the motor neurons in ALS eventually leads to their death. When the motor neurons die, the ability of the brain to initiate and control muscle movement is lost. With voluntary muscle action progressively affected, patients in the later stages of the disease may become totally paralyzed.

A “polyglutamine expansion neurodegenerative disease” or “polyQ expansion neurodegenerative disease” is a neurodegenerative disease or disorder which is caused by CAG repeat expansions in the gene, encoding polyglutamine (polyQ) stretches in the corresponding protein.

“Intermediate length polyglutamine expansions” comprise between 24-34 glutamines, more preferably between 24 to 33 glutamines.

Ataxin-2, encoded by the ATXN2 gene, is a polyglutamine protein which is mutated in spinocerebellar ataxia type 2 (SCA2) and in ALS. The protein sequence for human ataxin-2 is provided at GenBank. Accession No. NP 002964.3. Also see U.S. Pat. No. 6,844,431 which describes isolated SCA2 nucleic acids, isolated protein and methods of use thereof.

A “proteinopathy” is a disease which is characterized by accumulation of toxic insoluble protein aggregates in cells. Exemplary disorders, include, without limitation, ALS, FTD, FTLD-U, Alzheimer's disease, Huntington's disease, Parkinson's disease, and other motor neuron diseases.

Agents which modulate “TDP-43 mediated cellular toxicity” are those agents which affect at least one of cellular viability, morphology, aberrant protein aggregation, and replication in the presence of TDP-43 or functional variants thereof.

When the terms “prevent,” “preventing,” or “prevention” are used herein in connection with a given treatment for ALS, they mean that the treated subject either does not develop a clinically observable level ALS at all, or the condition develops more slowly and/or to a lesser degree in the subject than it would have absent the treatment. These terms are not limited solely to a situation in which the subject experiences no aspect ALS whatsoever. For example, a treatment will be said to have “prevented” ALS if it is given to a subject at risk of developing a ALS and results in the subject's experiencing fewer and/or milder symptoms of the proteinopathy than otherwise expected. A treatment can “prevent” ALS when the subject displays only mild overt symptoms of ALS. “Prevention” does not imply that there must have been no symptoms of ALS in any cell of a subject.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the TDP-43 or genetic modifier encoding nucleic acid molecule such that the latter is transcribed into mRNA. Host cell machinery then translates mRNA into a polypeptide. As mentioned hereinbelow, a variety of transgenic organisms are contemplated for use in the screening assays of the invention.

Those skilled in the art will recognize that a nucleic acid vector can contain nucleic acid elements other than the promoter element and the genetic modulator encoding nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, zebrafish, worm, insect and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

### Methods of Using Nucleic Acids Encoding Ataxin-2 Intermediate Length Polyglutamine Expansions in Assays for Diagnosing an Increased Risk of Early Onset ALS

The identification of ataxin-2 molecules comprising intermediate length polyglutamine (polyQ) expansions and their association with ALS facilitates the development of a diagnostic assay for an increased risk of developing early onset ALS. Ataxin-2 intermediate length polyglutamine (polyQ) expansion containing nucleic acids, including those described in Example I may be used for a variety of purposes in accordance with the present invention. Ataxin-2 intermediate length polyglutamine (polyQ) expansion containing DNA, RNA, or fragments thereof may be used as probes to detect the presence of and/or expression of the same in patient samples. Nucleic acids comprising ataxin-2 intermediate length polyglutamine (polyQ) expansions may be utilized as probes for such assays including but are not limited to: (1) in situ hybridization; (2) Southern hybridization (3) northern hybridization; and (4) assorted amplification reactions such as polymerase chain reactions (PCR).

Further, assays for detecting ataxin-2 intermediate length polyglutamine (polyQ) expansion may be conducted on any type of biological sample, including but not limited to body fluids (including blood, CNS fluid, urine, serum, gastric lavage), any type of cell (such as brain cells, white blood cells, mononuclear cells) or body tissue.

In most embodiments for screening for the presence of nucleic acids encoding ataxin-2 comprising intermediate length polyglutamine (polyQ) expansions, nucleic acid in the sample will initially be amplified, e.g. using PCR, to increase the amount of the templates as compared to other sequences present in the sample. This allows the target sequences to be detected with a high degree of sensitivity if they are present in the sample. This initial step may be avoided by using highly sensitive array techniques that are becoming increasingly important in the art. Alternatively, new detection technologies can overcome this limitation and enable analysis of small samples containing as little as 1 μg of total RNA. Using Resonance Light Scattering (RLS) technology, as opposed to traditional fluorescence techniques, multiple reads can detect low quantities of mRNAs using biotin labeled hybridized targets and anti-biotin antibodies. Another alternative to PCR amplification involves planar wave guide technology (PWG) to increase signal-to-noise ratios and reduce background interference. Reagents for performing both techniques are commercially available from Qiagen Inc. (USA). Also encompassed by the present invention are methods for high throughput sequencing DNA isolated from patients. Such methods are well known to those of skill in the art.

Methods for detecting CAG repeats in target nucleic acids have been previously described. Such methods can be modified for detection of CAG repeats of varying lengths. Methods for detection of large expansions (>100) are described in Cagnoli et al. (2006) J. Mol. Design 8:128-132. A Rapid Touch Down PCR assay for detection of polyglutamine expansions in SCA1, 2, 3, 6 and 7 has been described by Condorelli et al. (1998) Int. J. Clin. Lab. Res. 28:174-178. Also see Nat. Genet. (1996), 3:269-76 and Nat. Genet. (1996), 3:277-84 and U.S. Pat. No. 6,673,535 which provides reagents and methods suitable for detection CAG repeats in SCA2 protein. In one embodiment, ataxin-2 trinucleotide repeat size determinations on patient samples will be performed as set forth below in the methods section. Each of the aforementioned citations is incorporated herein by reference. Other assays for detecting CAG repeats are commercially available from Athena Diagnostics.

In an alternative approach, antibodies immunologically specific for ataxin-2 containing intermediate length polyQ expansions are developed using conventional methods. Such antibodies could then be used to advantage in immunhistochemistry or FACS assays to identify cells expressing aberrant polyQ containing ataxin-2 associated with the development of ALS.

### Kits and Articles of Manufacture

Any of the aforementioned products can be incorporated into a kit which can contain nucleic acids encoding ataxin-2 comprising intermediate length polyglutamine (polyQ) expansions or other such markers immobilized on a Gene Chip, PCR primers such as those described herein below. One embodiment of the kit comprises primers (e.g., SEQ ID NO: 1 and SEQ ID NO: 2) and reagents suitable for performance of PCR. Other reagents can include oligonucleotides, ataxin-2 polypeptides with and without polyQ expansions for use as controls, an antibody, a label, marker, or reporter, a pharmaceutically acceptable carrier, instructions for use, a container, a vessel for administration, an assay substrate, or any combination thereof.

### Screening Assays for Identifying Agents which Modulate Pathological Complex Formation Between Ataxin-2 Comprising Intermediate Polyglutamine Expansions and TDP-43 for Identifying Agents Having Efficacy for the Treatment of ALS

The methods described herein include methods (also referred to herein as “screening assays”) for identifying compounds that modulate (i.e., increase or decrease) complex formation between aberrant ataxin-2 and TDP-43. Such compounds include, e.g., polypeptides, peptides, antibodies, peptidomimetics, peptoids, small inorganic molecules, small non-nucleic acid organic molecules, nucleic acids (e.g., anti-sense nucleic acids, siRNA, oligonucleotides, synthetic oligonucleotides), carbohydrates, or other agents that bind to the target proteins and have a stimulatory or inhibitory effect thereon. Compounds thus identified can be used to modulate the expression or activity of ataxin-2 and/or TDP43 proteins in a therapeutic protocol.

In general, screening assays involve assaying the effect of a test agent on expression or activity of a target nucleic acid or target protein in a test sample (i.e., a sample containing the target nucleic acid or target protein). Expression or activity in the presence of the test compound or agent can be compared to expression or activity in a control sample (i.e., a sample containing the target protein that is incubated under the same conditions, but without the test compound). A change in the expression or activity of the target nucleic acid or target protein in the test sample compared to the control indicates that the test agent or compound modulates expression or activity of the target nucleic acid or target protein and is a candidate agent.

Compounds to be screened or identified using any of the methods described herein can include various chemical classes, though typically small organic molecules having a molecular weight in the range of 50 to 2,500 daltons. These compounds can comprise functional groups necessary for structural interaction with proteins (e.g., hydrogen bonding), and typically include at least an amine, carbonyl, hydroxyl, or carboxyl group, and preferably at least two of the functional chemical groups. These compounds often comprise cyclical carbon or heterocyclic structures and/or aromatic or polyaromatic structures (e.g., purine core) substituted with one or more of the above functional groups.

In alternative embodiments, compounds can also include biomolecules including, but not limited to, peptides, polypeptides, peptidomimetics (e.g., peptoids), amino acids, amino acid analogs, saccharides, fatty acids, steroids, purines, pyrimidines, derivatives or structural analogues thereof, polynucleotides, nucleic acid aptamers, and polynucleotide analogs. Compounds can be identified from a number of potential sources, including: chemical libraries, natural product libraries, and combinatorial libraries comprised of random peptides, oligonucleotides, or organic molecules. Chemical libraries consist of diverse chemical structures, some of which are analogs of known compounds or analogs or compounds that have been identified as “hits” or “leads” in other drug discovery screens, while others are derived from natural products, and still others arise from non-directed synthetic organic chemistry. Natural product libraries re collections of microorganisms, animals, plants, or marine organisms which are used to create mixtures for screening by: (1) fermentation and extraction of broths from soil, plant or marine microorganisms, or (2) extraction of plants or marine organisms. Natural product libraries include polypeptides, non-ribosomal peptides, and variants (non-naturally occurring) thereof. For a review, see Science 282:63-68 (1998). Combinatorial libraries are composed or large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of test compounds through the use of the various libraries herein permits subsequent modification of the test compound “hit” or “lead” to optimize the capacity of the “hit” or “lead” to prevent or suppress aberrant TDP-43-ataxin-2 complex formation.

In one embodiment, assays are provided for screening candidate or test molecules that are substrates of a target protein or a biologically active portion thereof in a cell. In another embodiment, the assays are for screening candidate or test compounds that disrupt complex formation between TDP-43 and ataxin-2.

In one embodiment, a cell-based assay is employed in which a cell, such as the yeast cells described in Example I, is contacted with a test compound. The ability of the test compound to modulate complex formation between ataxin-2 and TDP-43 and resulting cellular toxicity is then determined. Other cells of mammalian origin, e.g., rat, mouse, or human are also suitable for this purpose.

The ability of the test compound to bind to a target protein (e.g., TDP-43 or ataxin-2) or modulate target protein binding to a compound, e.g., a target protein substrate, can also be evaluated. This can be accomplished, for example, by coupling the compound, e.g., the substrate, with a radioisotope or enzymatic label such that binding of the compound, e.g., the substrate, to the target protein can be determined by detecting the labeled compound, e.g., substrate, in a complex. Alternatively, the target protein can be coupled with a radioisotope or enzymatic label to monitor the ability of a test compound to modulate target protein binding to a target protein substrate in a complex. For example, compounds (e.g., target protein substrates) can be labeled with¹²⁵1, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound to interact with target protein with or without the labeling of any of the interactants can be evaluated. For example, a microphysiometer can be used to detect the interaction of a compound with a target protein without the labeling of either the compound or the target protein (McConnell et al., Science 257:1906-1912, 1992). As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between a compound and a target protein.

In yet another embodiment, a cell-free assay is provided in which a target protein or biologically active portion thereof is contacted with a test compound and the ability of the test compound to bind to the target protein or biologically active portion thereof is evaluated. In general, biologically active portions of target proteins to be used in assays described herein include fragments that participate in interactions with other molecules, e.g., fragments with high surface probability scores.

Cell-free assays involve preparing a reaction mixture of the target proteins and the test compound under conditions and for a time sufficient to allow the two components to interact and bind, thus forming a complex that can be removed and/or detected. The ability of a target protein to bind to a target molecule can be determined using real-time Biomolecular Interaction Analysis (BIA) (e.g., Sjolander et al., Anal. Chem., 63:2338-2345, 1991, and Szabo et al., Curr. Opin. Struct. Biol., 5:699-705, 1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In several of these assays, the target proteins or the test substance is anchored onto a solid phase. The target protein/test compound complexes anchored on the solid phase can be detected at the end of the reaction. Generally, the target proteins are anchored onto a solid surface, and the test compound (which is not anchored) can be labeled, either directly or indirectly, with detectable labels discussed herein. It may be desirable to immobilize either the target protein, an anti-target protein antibody, or its target molecule to facilitate separation of complexed from uncomplexed forms of one or both of the proteins, as well as to accommodate automation of the assay. Binding of a test compound to a target protein, or interaction of a target protein with a target molecule in the presence and absence of a test compound, can be accomplished in any vessel suitable for containing the reactants. Examples of such vessels include microliter plates, test tubes, and micro-centrifuge tubes. In one embodiment, a fusion protein can be provided that adds a domain that allows one or both of the proteins to be bound to a matrix. For example, glutathione-S-transferase/target protein fusion proteins or glutathione-S-transferase/target fusion proteins can be adsorbed onto glutathione Sepharose™ beads (Sigma Chemical, St. Louis, Mo.) or glutathione derivatized microtiter plates, which are then combined with the test compound or the test compound and either the non-adsorbed target protein. The mixture is then incubated under conditions conducive to complex formation (e.g., at physiological conditions for salt and pH). Following incubation, the beads or microtiter plate wells are washed to remove any unbound components, the matrix immobilized in the case of beads, and the complex determined either directly or indirectly, for example, as described above.

Alternatively, the complexes can be dissociated from the matrix, and the level of target protein binding or activity determined using standard techniques.

Other techniques for immobilizing a target protein on matrices include using conjugation of biotin and streptavidin. Biotinylated target protein can be prepared from biotin-NHS (N-hydroxy-succinimide) using techniques known in the art (e.g., biotinylation kit, Pierce Chemicals, Rockford, IU.), and immobilized in the wells of streptavidin-coated 96 well plates (Pierce Chemical).

To conduct the assay, the non-immobilized component is added to the coated surface containing the anchored component. After the reaction is complete, unreacted components are removed (e.g., by washing) under conditions such that any complexes formed will remain immobilized on the solid surface. The complexes anchored on the solid surface can be detected in a number of ways. Where the previously non-immobilized component is pre-labeled, the presence of a label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In some cases, the assay is performed utilizing antibodies reactive with target protein, but which do not interfere with binding of the target protein to its target molecule. Such antibodies can be derivatized to the wells of the plate, and unbound target protein trapped in the wells by antibody conjugation. Methods for detecting such complexes, in addition to those described above for the GST-immobilized complexes, include immunodetection of complexes using antibodies reactive with the target protein or target molecule, as well as enzyme-linked assays which rely on detecting an enzymatic activity associated with the target protein. Alternatively, cell-free assays can be conducted in a liquid phase. In such an assay, the reaction products are separated from unreacted components, by any of a number of standard techniques, including but not limited to: differential centrifugation (see, for example, Rivas and Minton, Trends Biochem. Sci., 18:284-7, 1993); chromatography (gel filtration chromatography, ion-exchange chromatography); electrophoresis (e.g., Ausubel et al., eds. Current Protocols in Molecular Biology 1999, J. Wiley: New York.); and immunoprecipitation (see, for example, Ausubel et al., eds., 1999, Current Protocols in Molecular Biology, J. Wiley: New York). Such resins and chromatographic techniques are known to one skilled in the art (e.g., Heegaard, J. MoI. Recognit, 11: 141-148, 1998; Hage et al., J. Chromatogr. B. Biomed. Sci. Appl, 699:499-525, 1997). Further, fluorescence energy transfer may also be conveniently utilized, as described herein, to detect binding without further purification of the complex from solution. The assay can include contacting the target protein or a biologically active portion thereof with a known compound that binds to the target protein to form an assay mixture, contacting the assay mixture with a test compound, and determining the ability of the test compound to interact with the target protein, wherein determining the ability of the test compound to interact with the target protein includes determining the ability of the test compound to preferentially bind to the target protein or biologically active portion thereof, or to modulate the activity of a target molecule, as compared to the known compound.

A target protein can, in vivo, interact with one or more cellular or extracellular macromolecules, such as proteins. For the purposes of this discussion, such cellular and extracellular macromolecules are referred to herein as “binding partners.” Compounds that disrupt such interactions are useful for regulating the activity thereof. Such compounds can include, but are not limited, to molecules such as antibodies, peptides, and small molecules. In general, target proteins for use in identifying agents that disrupt interactions are the target proteins identified herein. To identify compounds that interfere with the interaction between the target protein and its binding partner(s), a reaction mixture containing the target protein and the binding partner is prepared, under conditions and for a time sufficient, to allow the two products to form a complex. To test an inhibitory agent, the reaction mixture is provided in the presence (test sample) and absence (control sample) of the test compound. The test compound can be initially included in the reaction mixture, or can be added at a time subsequent to the addition of the target gene and its cellular or extracellular binding partner. Control reaction mixtures are incubated without the test compound or with a control compound. The formation of complexes between the target protein and the cellular or extracellular binding partner is then detected. The formation of a complex in the control reaction, and less formation of complex in the reaction mixture containing the test compound, indicates that the compound interferes with the interaction of the target protein and the interactive binding partner. Such compounds are candidate compounds for inhibiting the expression or activity or a target protein. Additionally, complex formation within reaction mixtures containing the test compound and normal target protein can also be compared to complex formation within reaction mixtures containing the test compound and mutant target gene product. This comparison can be important in those cases wherein it is desirable to identify compounds that disrupt interactions of mutant but not normal target protein.

Binding assays can be carried out in a liquid phase or in heterogenous formats. In one type of heterogeneous assay system, either the target protein or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

To conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

In another embodiment, modulators of target expression (RNA or protein) are identified. For example, a cell or cell-free mixture is contacted with a test compound and the expression of target mRNA (e.g., ataxin-2 encoding mRNA) or protein evaluated relative to the level of expression of target mRNA or protein in the absence of the test compound. When expression of target mRNA or protein is greater in the presence of the test compound than in its absence, the test compound is identified as a stimulator (candidate compound) of target mRNA or protein expression. Alternatively, when expression of target mRNA or protein is less (statistically significantly less) in the presence of the test compound than in its absence, the test compound is identified as an inhibitor (candidate compound) of target mRNA or protein expression. The level of target mRNA or protein expression can be determined by methods described herein and methods known in the art such as Northern blot or Western blot for detecting target mRNA or protein.

In another aspect, the methods described herein pertain to a combination of two or more of the assays described herein. For example, a modulating agent can be identified using a cell-based or a cell-free assay, and the ability of the agent to modulate the activity of a target protein can be confirmed in vivo, e.g., in an animal such as an animal model for ALS.

This invention further pertains to novel agents identified by the above-described screening assays. Accordingly, it is within the scope of this invention to further use an agent (compound) identified as described herein (e.g., a target protein modulating agent, an anti sense nucleic acid molecule, an siRNA, a target protein-specific antibody, or a target protein-binding partner) in an appropriate animal model to determine the efficacy, toxicity, side effects, or mechanism of action, of treatment with such an agent. Furthermore, novel agents identified by the above-described screening assays can be used for treatments as described herein.

Compounds that modulate target protein expression or activity (target protein modulators) can be tested for their ability to affect metabolic effects associated with the target protein, e.g., with decreased expression or activity of target protein using methods known in the art and methods described herein. For example, the ability of a compound to modulate ataxin-2/TDP-43 complex formation and associated toxicity can be tested using an in vitro or in vivo model for ALS.

The compounds identified above can be synthesized by any chemical or biological method. The compounds identified above can also be pure, or may be in a heterologous composition (e.g., a pharmaceutical composition), and can be prepared in an assay-, physiologic, or pharmaceutically-acceptable diluent or carrier (see below).

### Pharmaceutical Compositions

A compound that is found to prevent or suppress aberrant TDP-43-ataxin-2 complex formation and cytotoxicity in a cell can be formulated as a pharmaceutical composition, e.g., for administration to a subject to treat ALS.

A pharmaceutical composition typically includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge et al., J. Pharm. Sci. 66:1-19, 1977).

The compound can be formulated according to standard methods. Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical. Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical. Association, 3rd ed. (2000) (ISBN: 091733096X). In one embodiment, a compound that prevents or suppresses aberrant TDP-43-ataxin-2 complex formation and cytotoxicity in a cell can be formulated with excipient materials, such as sodium chloride, sodium dibasic phosphate heptahydrate, sodium monobasic phosphate, and a stabilizer. It can be provided, for example, in a buffered solution at a suitable concentration and can be stored at 2-8° C. The pharmaceutical compositions may be in a variety of forms. These include, for example, liquid, semi-solid and solid dosage forms, such as liquid solutions {e.g., injectable and infusible solutions), dispersions or suspensions, tablets, capsules, pills, powders, liposomes and suppositories. The preferred form can depend on the intended mode of administration and therapeutic application. Typically compositions for the agents described herein are in the form of injectable or infusible solutions.

Such compositions can be administered by a parenteral mode {e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating a compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of a compound plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the compound can be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978. A compound identified as one that prevents or suppresses aberrant TDP-43-ataxin-2 complex formation and cytotoxicity in a cell can be modified, e.g., with a moiety that improves its stabilization and/or retention in circulation, e.g., in blood, serum, or other tissues, e.g., by at least 1.5, 2, 5, 10, or 50 fold. The modified compound can be evaluated to assess whether it can reach treatment sites of interest.

For example, the compound can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, a compound can be conjugated to a water soluble polymer, e.g., a hydrophilic polyvinyl polymer, e.g., polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; and branched or unbranched polysaccharides. When the compound is used in combination with a second agent (e.g., any additional therapies for a proteinopathy such as a decongestant or Rilutek®), the two agents can be formulated separately or together. For example, the respective pharmaceutical compositions can be mixed, e.g., just prior to administration, and administered together or can be administered separately, e.g., at the same or different times as elaborated below.

The following materials and methods are provided to facilitate the practice of the present invention.

### Yeast Strains and Media

The strain used in the modifier screen was TDP-43, MATa canl-100 his3-11,15 leu2-3,112 trp1-1 ura3-1 ade2-1 pAG303Gal-TDP-43. The huntingtin and α-synuclein strains are described^{15,17}. The pbp1Δ strain was obtained by replacing the PBP1 coding region with a KanMX4 cassette in the BY4741 strain background. Colony PCR was used to verify correct gene disruption. Strains were manipulated and media prepared using standard techniques⁵⁰.

### Plasmids

The CEN and 2-micron galactose-inducible TDP-43 yeast expression plasmids are described²². The PBP1 expression plasmid was constructed by shuttling PBP1 from the Gateway entry vector (pDONR221) into pBY011, a CEN, URA3, galactose-inducible yeast expression plasmid¹⁷. The CFP-tagged PBP1 construct was made by shuttling PBP1 into pAG413GPD-CFP-ccdB, a CEN, HIS3, constitutive promoter yeast expression plasmid⁵¹. The 2-micron a-synuclein expression plasmid was as described¹⁶. Site-directed mutagenesis was performed with the QuickChange Multi kit (Stratagene). The ΔNLS-TDP-43-YFP construct was generated by mutating residues lysine 82, arginine 83, and lysine 84 to alanine¹¹. The TDP-43 5F→L constructs were generated by mutating phenylalanine residues 147, 149, 194, 229, and 231 to leucine³⁵. Mammalian expression vectors were generated by shuttling TDP-43-YFP, ΔNLS-TDP-43-YFP, TDP-43-(5F→L)-YFP, or ΔNLS-TDP-43-(5F→L)-YFP from pDONR221 into pcDNA 3.2 (Invitrogen).

### Yeast Transformation and Spotting Assays

The PEG/lithium acetate method was used to transform yeast with plasmid DNA⁵². For spotting assays, yeast cells were grown overnight at 30° C. in liquid media containing raffinose (SRaf/-Ura) until log or mid-log phase. Cultures were then normalized for OD₆₀₀, serially diluted and spotted onto synthetic solid media containing glucose or galactose lacking uracil, and were grown at 30° C. for 2-3 d.

### Yeast TDP-43 Toxicity Modifier Screen

PBP1, the yeast ortholog of human Ataxin-2, was isolated in a high-throughput yeast transformation screen similar to previous screens^{17,19,53}. 5,500 full-length yeast ORFs (Yeast FLEXGene collection, http://www.hip.harvard.edu/research/yeast_flexgene/) were transformed into a strain expressing TDP-43 integrated at the HIS3 locus. A standard lithium acetate transformation protocol was modified for automation and used by employing a BIOROBOT Rapidplate 96-well pipettor (Qiagen). Transformants were grown overnight in synthetic deficient media lacking uracil (SD-Ura) with glucose. Overnight cultures were inoculated into fresh SD-Ura media with raffinose and allowed to reach stationary phase. The cells were spotted onto SD-Ura+glucose and SD-Ura+galactose agar plates. Modifiers were identified on galactose plates after 2-3 days of growth at 30° C.

### Drosophila Experiments

Transgenic flies expressing human TDP-43, TDP-43-YFP, TDP-43.Q331K and TDP-43.Q331K-YFP were generated by standard techniques using the pIJAST vector. Other lines were obtained from public stock centers. The effects of untagged and YFP tagged proteins were generally similar but the YFP tagged proteins were expressed at a higher steady-state level and caused more severe effects. Some experiments were performed at 25° C., whereas others were performed at 29° C. for a stronger effect (the GAL4/UAS system drives expression more strongly at higher temperature), or for time considerations (lifespan of flies is shorter at 29° C.). Climbing and lifespan analyses were performed as described⁵⁴. Fly Ataxin-2 reagents are described⁵⁵.

### Co-Immunoprecipitation

FIEK293T cells were transfected with TDP-43-YFP fusion constructs using FuGene 6 (Roche) according to the manufacturer's instructions. After 48 h, cells were washed with PBS, trypsinized and collected by centrifugation. Cells were washed in ice-cold PBS containing protease inhibitor cocktail (Roche) then lysed in NP-40 lysis buffer (150 mM NaCl, 50 mM Tris, pH 8.0, 1% NP-40 and protease inhibitor). In the case of RNA digestion, lysates were treated with 200 μg/mlRNase A for 15 minutes (Qiagen). Lysates were clarified by centrifugation and pre-cleared with Protein A agarose (Invitrogen). Immunoprecipitation was performed by incubating with α-GFP rabbit polyclonal antibody (1:750 dilution; Abeam) for 2 h, then protein A agarose beads (50 μl) for 1 h. The beads were washed 3× with NP-40 lysis buffer and resuspended in 4×SDS sample buffer (40% Glycerol, 240 mM. Tris HCL pH 6.8, 8% SDS, 0.04% Bromophenol Blue, 5% β-mercaptoethanol).

### Immunoblotting

Lysates were boiled 5 min, then subjected to SDS/PAGE (4-12% gradient Bis-Tris, Invitrogen) and transferred to PVDF membrane (Invitrogen). Membranes were blocked 1 h in 5% non-fat dry milk at RT and then incubated 0/N in primary antibody at 4° C. Membranes were washed in PBS, then incubated in HRP-conjugated secondary antibody (1:5000) 1 h, then washed in PBST (PBS+0.1% Tween20). Proteins were detected with Immobilon Western Chemiluminescent HRP Substrate (Millipore) and visualized on Biomax MR film (Kodak). Primary antibodies were: a-GFP mouse polyclonal antibody (Roche), 1:1000; α-Ataxin-2 mouse antibody (BD), 1 :500.

### Immunofluorescence

HEK293T cells were washed in PBS and fixed in 4% paraformaldehyde 15 min, then washed in 1×PBS 4×. Cells were blocked for 1 h in blocking solution (2% Fetal Bovine Serum, 0.02% Triton X-100, 1×PBS), and then incubated 1 h in primary antibody at RT. Cells were then washed 3× in blocking solution, then incubated with secondary antibody 1 h RT. Cells were then washed with blocking solution and mounted in Vectashield mounting media with DAPI (Vector). Antibodies used were: α-Ataxin-2 mouse antibody (BD), 1:500 and Cy-3 conjugated α-mouse IgG (Invitrogen), 1:500. Cells were visualized by light microscopy.

### Immunohistochemistry

SCA2 patient brain tissue was embedded in polyethylene glycol and cut into 100 μm thick serial sections. All other sections were deparaffinized before pretreatment using heat antigen retrieval with Bull's Eye Decloaker (BioCare Medical). Endogenous peroxidase was then blocked with 3% hydrogen peroxide in PBS for 10 minutes. After washing with 0.1% PBST and blocking with 10% goat serum, 0.5% PBST for 30-60 minutes at 25° C. Sections were incubated with mouse anti-Ataxin-2 (1:500; BD Biosciences) or rabbit anti-TDP-43 (1:500; Proteintech Group) in 0.1% PBST overnight at 4° C. After washing with 0.1% PBST, sections were incubated with biotinylated goat anti-mouse or rabbit IgG (1:200; Vector Laboratories) for 1 hour at 25° C. After washing with 0.1% PBST, sections were then incubated with Vectastain ABC (Vector Laboratories) for 45 minutes. After washing with 0.1% PBST followed by 0.1 M Tris (pH 7.5) and 0.3M NaCl. Peroxidase activity was then detected with DAB (Sigma). Detailed immunohistochemistry protocols are available at http://www.uphs.upenn.edu/mcrc.

### Patient-Derived Lymphoblastoid Cell Culture and Ataxin-2 Protein Stability

Lymphoblastoid cell lines were obtained from patients with ALS or unaffected normal controls (Coriell) and cultured in RPMI 1640 medium supplemented with 2 mM L-glutamine, 15% fetal bovine serum, penicillin and streptomycin. To assess Ataxin-2 stability, protein synthesis was inhibited by treating cells with cycloheximide (0.5 μM) for 0, 16 or 24 hours. Cells were washed once in PBS and lysed in ice-cold NP-40 lysis buffer containing protease inhibitors. Lysates were cleared by centrifugation and then suspended in 4× sample buffer and subjected to SDS/PAGE followed by immunoblotting for Ataxin-2 and b-Actin.

### Ataxin-2 Trinucleotide Repeat Size Determination in ALS Patients and Controls was Performed

Genomic DNA from human ALS patients was obtained from the Coriell. Institute for Medical Research (Coriell) or the Center for Neurodegenerative Disease Research (CNDR) at the University of Pennsylvania. 454 ALS samples from Coriell were distributed in 96-well plates NDPT103, NDPT026, NDPT025, NDPT100, NDPT030, and NDPT106. 100 additional ALS cases unselected for family history and a subsequent cohort of 23 FALS cases were obtained from the CNDR. CNDR ALS samples were verified to meet E1 Escorial criteria for definite or probable ALS. In addition, 80/123 CNDR ALS samples were neuropathologically confirmed to have ALS pathology with TDP-43 immunopositivity, while the remainder was from living patients. Among the original CNDR cohort of 100 cases unselected for family history, 13 cases (13%) are known to have a first or second-degree relative with ALS, in line with published estimates of ˜10% FALS. Clinical details were collected from 65/100 CNDR ALS cases unselected for family history via chart review by a neurologist; these details included age of onset, age of death, disease duration, gender, presence/absence of family history, and ALS functional rating scale score (ALS-FRS) at the time of initial neurological evaluation. Mutations SOD1 or TARDBP were excluded from the 23 additional CNDR cases of FALS. 286 neurologically normal control samples from Coriell were distributed in 96-well plates NDPT095, NDPT096, NDPT098, and NDPT099. One control sample (ND12820 from plate NDPT096) was excluded because of a documented family history of motor neuron disease; sibling ND12819 was diagnosed with progressive bulbar palsy. An additional 82 neurologically normal control samples were obtained from the Children's Hospital of Philadelphia.

We amplified Ataxin-2 CAG repeats from individual samples by polymerase chain reaction (PCR). PCR primers used for amplification were designed to amplify the CAG repeat region of human Ataxin-2 (bp 442-598). The 5′ primer was SCA2-Anew: 5′-CCC CGC CCG GCG TGC GAG CCG GTG TAT G-3′ (SEQ ID NO: 1). The 3′ primer was SCA2-B: 5′-CGG GCT TGC GGA CAT TGG-3′ (SEQ ID NO: 2). PCR cycles were as follows: 2 min 94° C., 35 cycles (1 min 94° C., 1 min 60° C., 1 min 72° C.), and 5 min 72° C. Initially, PCR products were resolved on a 2% agarose gel by electrophoresis, amplicons purified and cloned into the PCRII TA vector (Invitrogen), and repeat lengths were determined by DNA sequencing. Subsequently, for large-scale analysis of Ataxin-2 CAG repeat lengths, a capillary electrophoresis approach was used, incorporating the 6FAM fluorophor into the PCR products in the 5′ SCA2-Anew primer. PCR products were mixed with Liz-500 size standard (Applied. Biosystems) and were processed for size determination on an. ABI3730 sequencer. The sizes of the repeats were determined with GeneMapper™ 4.0 software (Applied Biosystems). All 32 samples with repeat expansions were verified by independent PCR as above, followed by resolution on a 4% agarose gel, to confirm relative lengths and also by capillary electrophoresis. To further confirm repeat expansions, amplicons from 21 of 32 samples were cloned and sequenced.

### Statistical Analyses

Two-tailed T tests were used to compare age of onset, ALS-FRS at the time of initial neurological evaluation, and age of death in ALS with and without intermediate-length Ataxin-2 repeats after ascertainment that distributions met assumptions of normality. Disease durations for the two groups, which were not distributed normally, were compared using Mann-Whitney tests. Two-tailed. Fisher's exact tests were used to compare gender and presence/absence of family history between the two groups. For all tests, percentages and statistical testing were calculated based only on the cases for which relevant clinical data were available.

Two-tailed Fisher's exact tests were used to evaluate genetic association between intermediate-length Ataxin-2 repeats and ALS, and odds ratios were calculated, under an intermediate-length Ataxin-2 repeat-dominant model.

The following examples are provided to illustrate certain embodiments of the invention. They are not intended to limit the invention in any way.

### Example I

### Ataxin-2 Intermediate-Length Polyglutamine Expansions are Associated with Amyotrophic Lateral Sclerosis (ALS)

The data presented herein demonstrate that TDP-43 and ataxin-2 associate in a complex and are mislocalized in ALS patient spinal cord neurons. Given that Ataxin-2 is a polyQ disease gene, we analyzed the length of the polyQ repeat in over 600 sporadic and familial ALS patients, and found a significant association of Ataxin-2 intermediate-length polyQ tract expansions with ALS (4.5% of cases). Remarkably, the presence of intermediate-length polyQ tract expansions in Ataxin-2 was significantly associated with a >10 year advanced age of ALS onset.

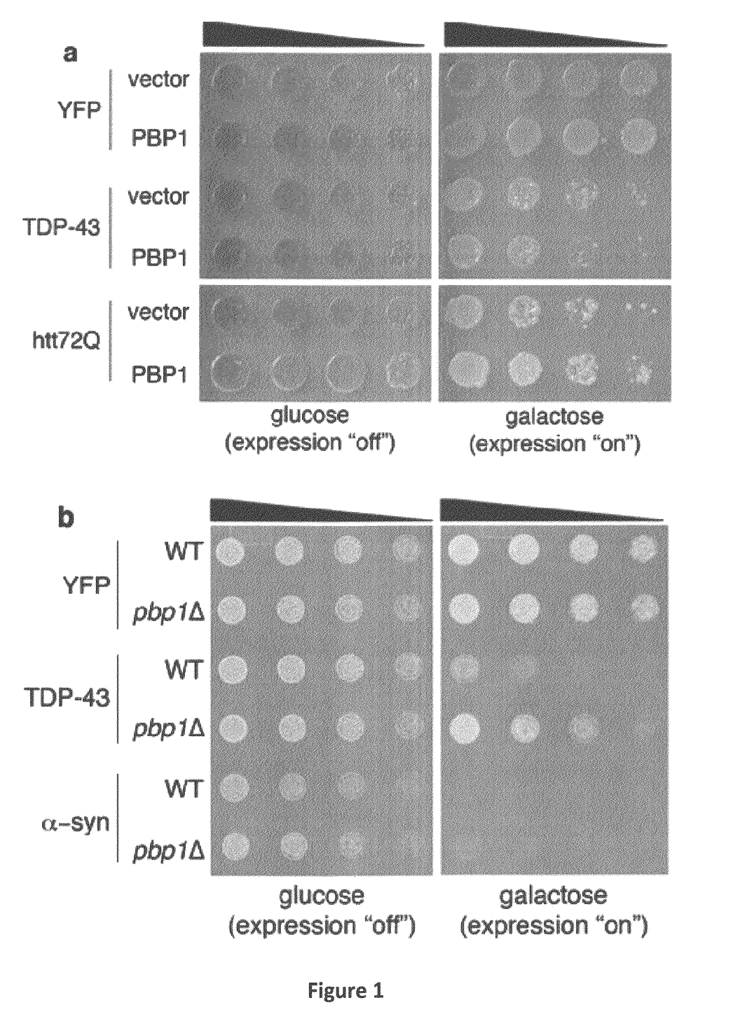

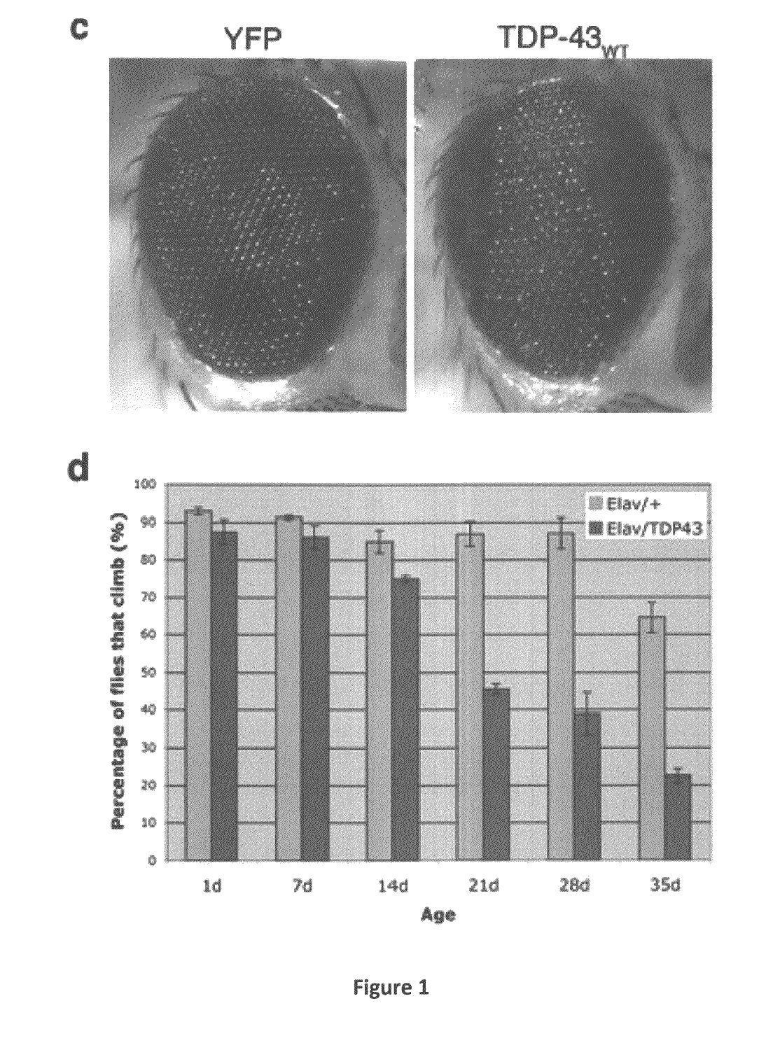

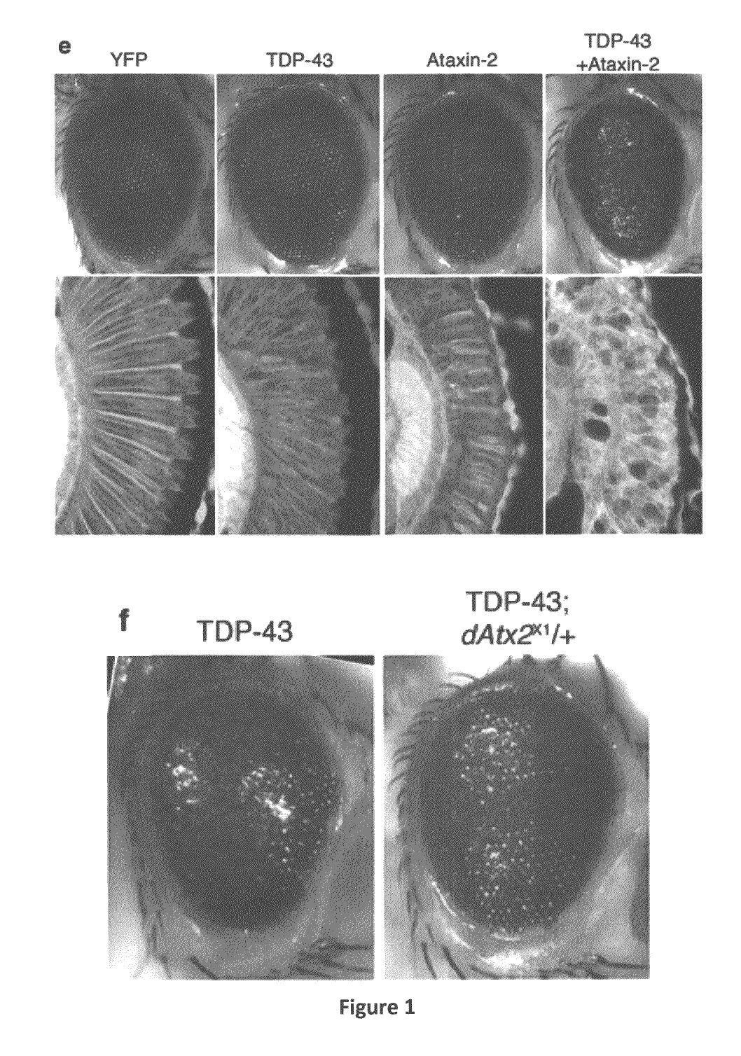

To gain insight into mechanisms of TDP-43 pathogenesis, we used an unbiased genetic approach to identify genes that could suppress or enhance TDP-43 toxicity in yeast. One yeast gene identified that enhanced TDP-43 toxicity, PBP1 (poly(A)-binding protein; (Pab1p)-Binding Protein), was notable as an ortholog of the human Ataxin-2 gene, mutations in which cause the neurodegenerative disease spinocerebellar ataxia type 2 (SCA2). SCA2 is one of a heterogeneous group of 28 autosomal dominant hereditary ataxias²⁴ and is caused by polyQ tract expansions in the Ataxin-2 gene (ATXN 2)²⁵⁻²⁸. Interestingly, in SCA2, as in ALS, motor neurons are also known to degenerate, but these features typically occur later than the cerebellar degeneration. However, in select cases, the motor neuron features of SCA2 are prominent enough to mimic an ALS presentation^{29,30}, indicating the potential for clinicopathological overlap. Although the precise functions of yeast Pbp1 and human Ataxin-2 are not fully understood, Pbp1 interacts with Pab1 to regulate mRNA polyadenylation and is involved in stress granule assembly³¹. P-bodies and stress granules play important roles in regulating translation, mRNA degradation, and the subcellular localization of mRNAs³². Upregulation of Pbp1 enhanced TDP-43 toxicity in yeast (FIG. 1a), whereas Pbp1 loss-of-function suppressed toxicity (FIG. 1b), indicating that Pbp1 is a dose-sensitive modifier of TDP-43 toxicity. Pbp1 upregulation did not enhance the toxicity of another human neurodegenerative disease protein, pathogenic Huntingtin (FIG. 1a), and Pbp1 deletion did not suppress toxicity of a-synuclein (FIG. 1b), demonstrating specificity of the Pbp1 interaction for TDP-43.

To test the relevance of the Ataxin-2/TDP-43 genetic interaction in the nervous system we first used Drosophila. Directing expression of WT or an ALS-linked mutant TDP-43 to the eye of the fly caused progressive, age-dependent, degeneration of the structure (FIG. 1c, 1g, 1h). Directing expression to the nervous system caused progressive loss of motility and reduced lifespan (FIG. 1d and data not shown). Upregulation of the fly homolog of Ataxin-2, dAtx2, enhanced toxicity of TDP-43, resulting in dramatically more severe retinal degeneration and a further shortened lifespan (FIG. 1e and data not shown). The effect of dAtx2 was dose-dependent as reducing levels of dAtx2 by 50% mitigated TDP-43 toxicity (FIG. 10, indicating that toxicity of TDP-43 is sensitive to the levels of dAtx2. The interaction was specific, as upregulation of the molecular chaperone Hsp70 did not modify TDP-43 toxicity (FIG. 1i), as it does in models of Parkinson's disease and spinocerebellar ataxia type 3 (SCA3)^{33,34}. These data indicate that modulation of TDP-43 toxicity by Ataxin-2 seen in yeast is conserved in the nervous system of the fly.

### TDP-43 and Ataxin-2 Interactions Depend on RNA Binding

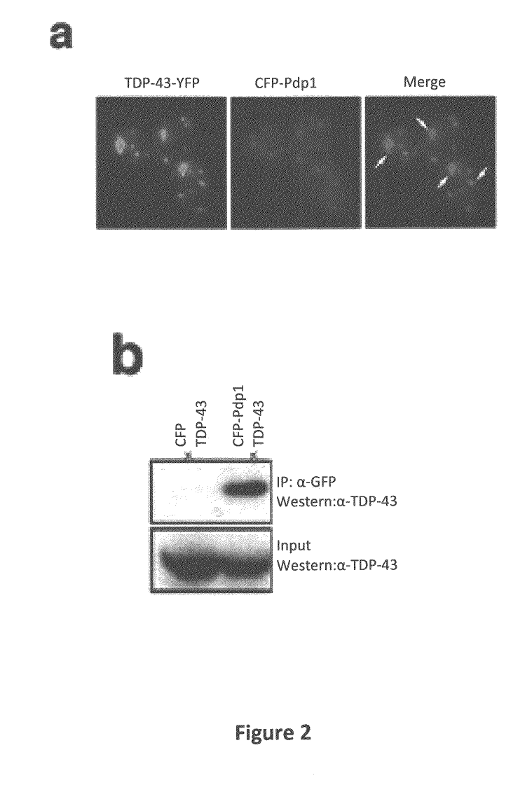

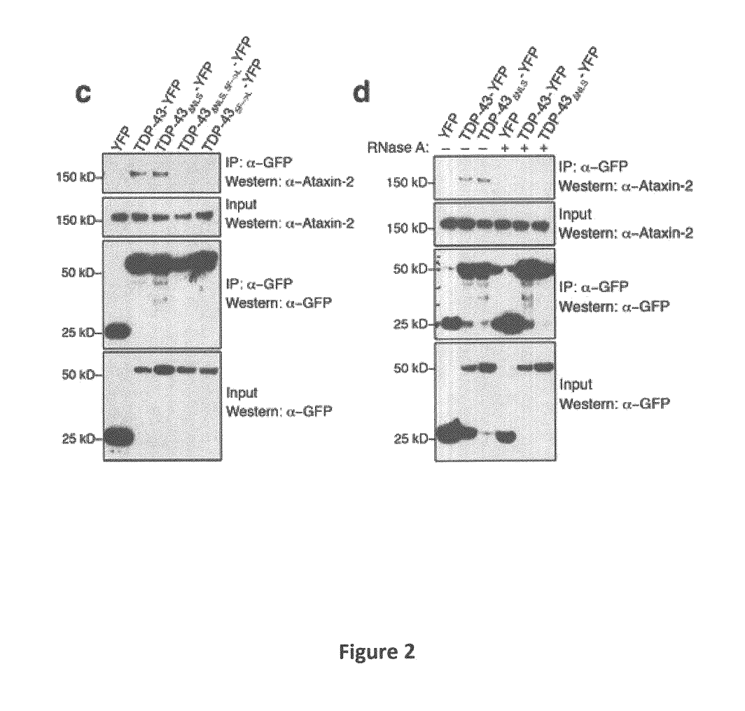

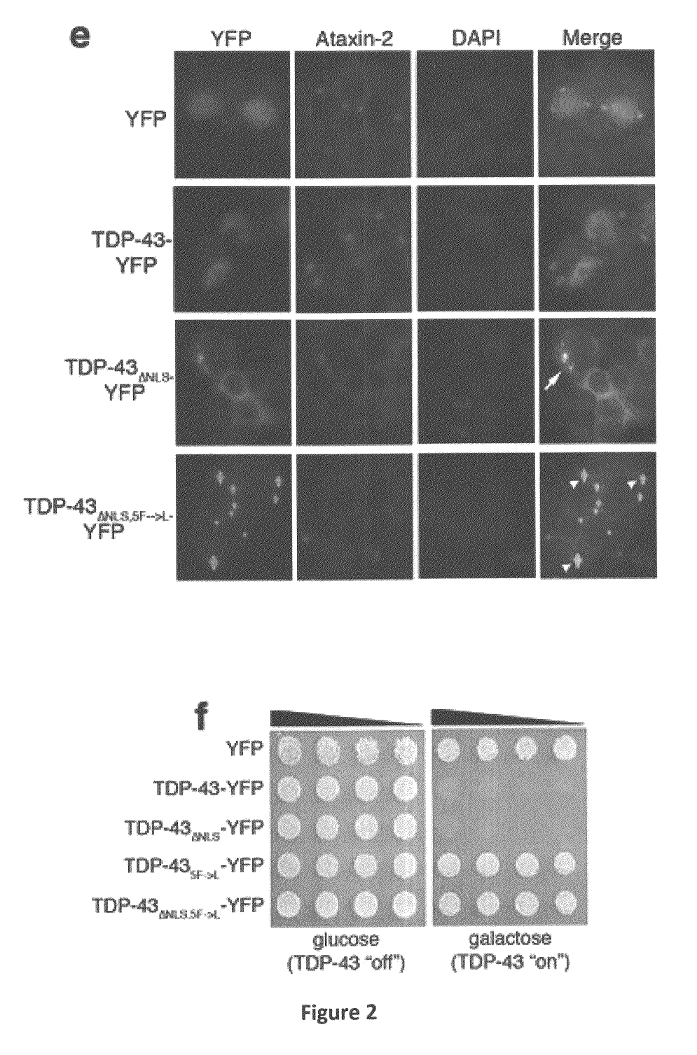

Given the striking effects of Ataxin-2 on TDP-43 toxicity in yeast and flies, we determined whether the two proteins could physically interact in yeast and human cells. Yeast cells were transformed with YFP-tagged TDP-43 and either CFP-tagged Pbp1 or CFP alone, and the localization of the proteins visualized by fluorescence microscopy. These studies showed that Pbp1 localized to TDP-43 cytoplasmic accumulations (FIG. 2a). To determine whether Pbp1 and TDP-43 could associate in the same protein complex, we performed immunoprecipitation assays with an antibody directed against the Pbp1 epitope tag followed by immunoblotting to detect TDP-43. These studies confirmed the ability of TDP-43 to interact with Pbp1 in the same complex (FIG. 2b). To determine whether this interaction was conserved in human cells, HEK293T cells were transfected with YFP-tagged TDP-43 or YFP alone. TDP-43-YFP, but not YFP alone, immunoprecipitated endogenous human Ataxin-2 (FIG. 2c). These data indicate that Ataxin-2 and TDP-43 form part of the same complex in both yeast and human cells.

Because both TDP-43 and Ataxin-2 are involved in RNA metabolism³⁵⁻³⁷, we considered that RNA binding may be important for the TDP-43/Ataxin-2 interaction. TDP-43 is an RRM-containing protein with highly conserved RNP-1 and RNP-2 consensus motifs in each RRM. Within these motifs, specific aromatic residues have been shown to be necessary for RNA base stacking interactions³⁸ and mutation of these residues (Phe to Leu) reduces the ability of TDP-43 to bind RNA in vitro³⁵. To address the significance of TDP-43 RNA binding, we mutated all 5 of these residues to generate a TDP-43_{(5F}_{_{→}}_{L)}-YFP protein, and determined the effect on the interaction with Ataxin-2. Mutation of the RRM domain abolished the ability of TDP-43 to interact with Ataxin-2 (FIG. 2c), suggesting that RNA likely serves as a bridge between the two proteins, although these aromatic residues within the RRMs could also contribute to protein-protein interactions. Therefore, to further demonstrate a role for RNA in mediating the TDP-43/Ataxin-2 interaction, we performed the immunoprecipitation with full-length WT protein in the presence of RNase. RNase treatment abolished the interaction between TDP-43 and Ataxin-2 (FIG. 2d). Finally, we transfected HEK293T cells with YFP-tagged WT and RRM-domain mutant TDP-43 constructs and immunostained for endogenous Ataxin-2 (FIG. 2e). Consistent with previous reports, Ataxin-2 was predominantly localized to the cytosol and occasionally formed punctate cytoplasmic accumulations³⁹. TDP-43-YFP remained mostly in the nucleus. However, a form of TDP-43 with a mutated nuclear localization signal (NLS; ΔNLS-TDP-43-YFP) to restrict TDP-43 to the cytosol, which is its presumed pathogenic localization in disease¹¹, remained in the cytosol where it occasionally formed aggregates (FIG. 2e); these aggregates always co-localized with Ataxin-2 (FIG. 2e). However, mutating the RRM domains of TDP-43 in the context of the ΔNLS construct also resulted in TDP-43 aggregation in the cytosol, but these accumulations never co-localized with Ataxin-2 (FIG. 2e). In addition to blocking the interaction between TDP-43 and Ataxin-2, mutating the RRMs of TDP-43 also eliminated TDP-43 toxicity (FIG. 20. Taken together, these data indicate that TDP-43 and Ataxin-2 can interact in a complex in the cytoplasm—the site of toxic function of TDP-43 in disease—and that this interaction likely depends on RNA binding.

### Ataxin-2 Localization is Perturbed in ALS Patient Spinal Cord Neurons

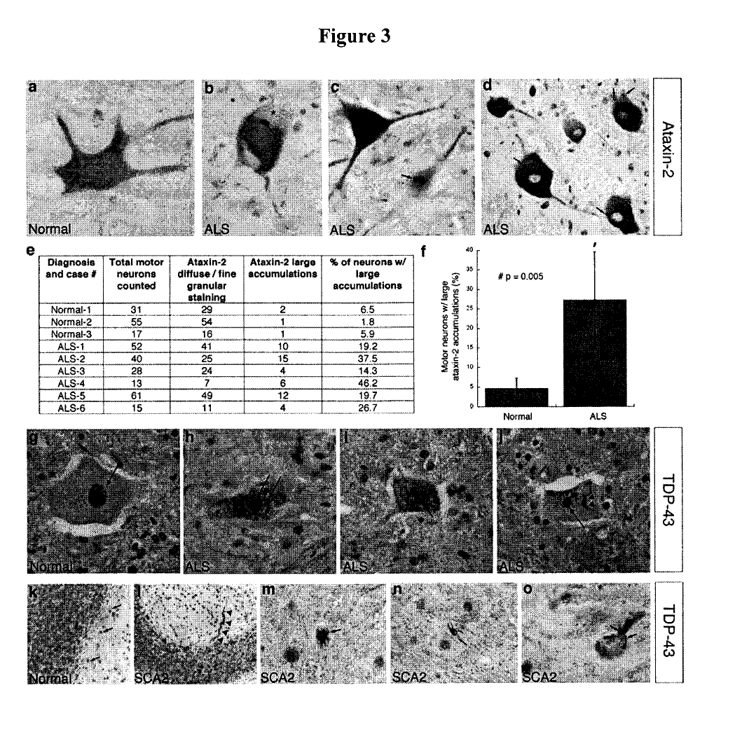

The genetic interactions between TDP-43 and Ataxin-2 in yeast and Drosophila, and the physical association in yeast and mammalian cells, suggested that Ataxin-2 might show abnormal localization in human disease. To address this, we examined Ataxin-2 localization in spinal cord neurons from six ALS patients and three neurologically normal controls (FIG. 3). Normally, Ataxin-2 is localized in a diffuse or fine-granular pattern throughout the cytoplasm of spinal cord neurons (FIG. 3a). However, in ALS spinal cord neurons, Ataxin-2 localization was altered, showing more distinct cytoplasmic accumulations (27% of ALS spinal cord neurons vs. 5% of control neurons in blinded-analysis, FIG. 3b-f). This pattern is strikingly similar to that of TDP-43 “pre-inclusions” seen in ALS (⁴⁰ and FIG. 3h,i), which, in contrast to the normal TDP-43 nuclear localization (FIG. 3g), form in the cytoplasm prior to apparent coalescence to larger round or skein-like inclusions (FIG. 3j). These studies indicate that Ataxin-2 localization is altered in spinal cord neurons of ALS patients.

### TDP-43 is Mislocalized to the Cytoplasm and Aggregated in SCA2 Patient Neurons

To further address the significance of interactions between TDP-43 and Ataxin-2, we examined the localization of TDP-43 in SCA2 patient tissue. Although a rare disease, we obtained tissue from two SCA2 patients and examined the cerebellum and brain stem nuclei for TDP-43 pathology. Normally, TDP-43 was restricted to the nucleus of cerebellar Purkinje neurons (FIG. 3k); however, in SCA2 tissue, surviving Purkinje cell neurons displayed abundant TDP-43 immunoreactivity in the cytoplasm with typical corkscrew-shape neurodegenerative signs (FIG. 31). TDP-43 immunoreactive inclusions were also present in motor neurons of the abducens and the hypoglossus nucleus, and in noradrenergic afferent neurons of the locus coeruleus nucleus within the brainstem (FIG. 3m-o). These data indicate that TDP-43 proteinopathy occurs in SCA2, and that TDP-43 and Ataxin-2 interactions could play important roles in the pathogenesis of both ALS and SCA2. This finding also provides a molecular explanation for the observed clinicopathological similarities between the two diseases^{29,30}.

Intermediate-Length polyQ Repeat Expansions in Ataxin-2 are Linked to ALS

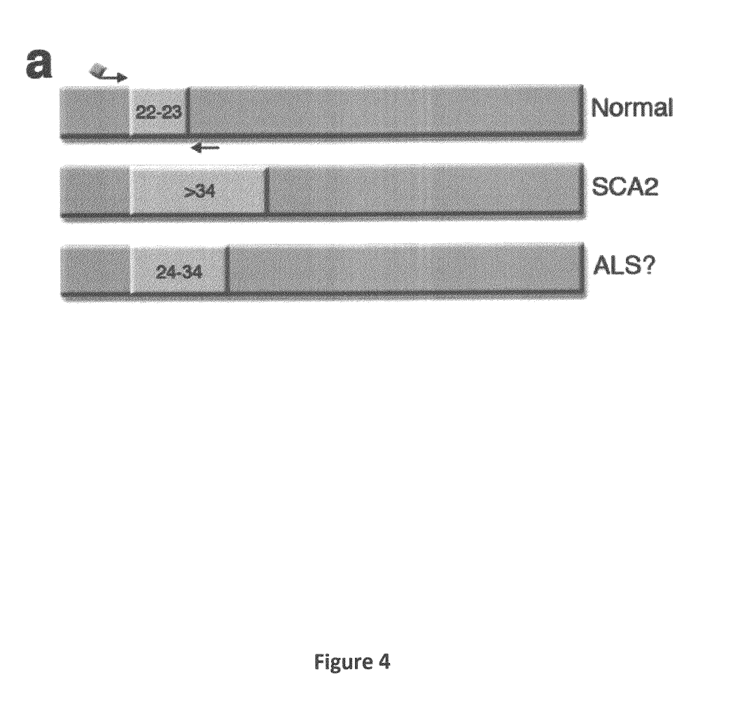

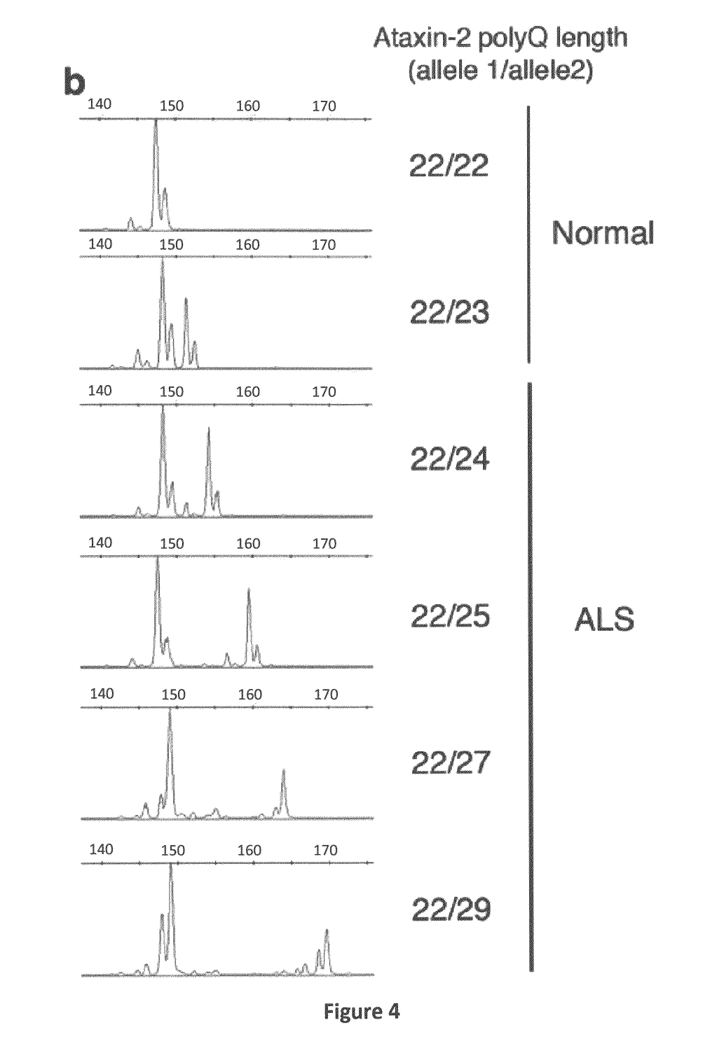

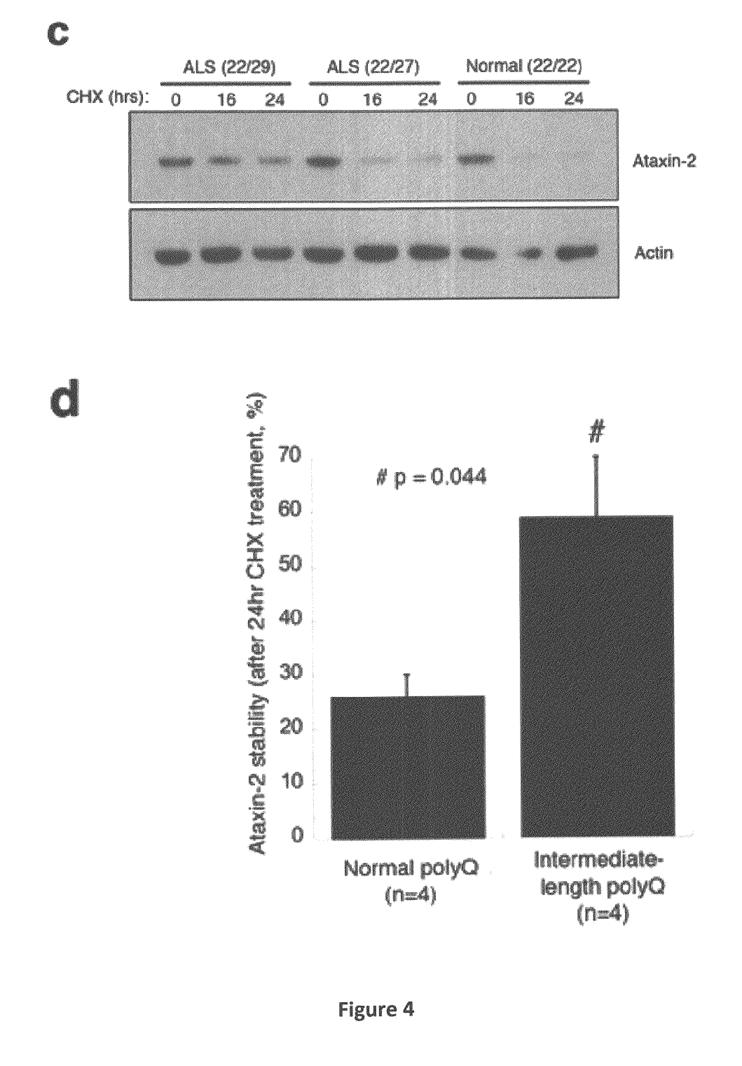

These genetic, biochemical, and neuropathological interactions between Ataxin-2 and TDP-43 raised the possibility that mutations in Ataxin-2 could play a causative role in ALS. The Ataxin-2 polyQ tract length, though variable, is most frequently 22-23, with expansions of >34 causing SCA2²⁵⁻²⁸. However, the variable nature of the Ataxin-2 repeat suggested a mechanism by which such mutations in Ataxin-2 could be linked to ALS: we hypothesized that intermediate-length repeat expansions greater than 23 but below the threshold for SCA2 may be associated with ALS (FIG. 4a). To test this, we defined the Ataxin-2 polyQ repeat length in genomic DNA from 554 individuals diagnosed with ALS and 368 neurologically normal controls (FIG. 4b, Table 1). Only 2 of 368 control cases (0.5%) were found to harbor a single intermediate-length Ataxin-2 allele each (repeat lengths of 24 and 26, respectively), while 25 of 554 ALS cases (4.5%) possessed one allele with an intermediate-length Ataxin-2 repeat (mean repeat length 28, range 24-33). Thus, intermediate-length Ataxin-2 polyQ repeat expansions are significantly associated with ALS (Table 1, p=2.1×10⁻⁴, odds ratio 8.6).

TABLE 1
Increased frequency of intermediate-length Ataxin-2 polyQ
repeat expansions in ALS.

| | | ≦23 | 24-32 | % 24-32 | P-value | Odds |
|---|---|---|---|---|---|---|
| | Total | Repeats | Repeats | Repeats | (Fisher's) | Ratio |
| ALS | 554 | 529 | 25 | 4.5% | 2.1 × 10⁻⁴ | 8.6 |
| unselected | | | | | | |
| for family | | | | | | |
| history | | | | | | |
| Familial | 23 | 20 | 3 | 13% | 1.7 × 10⁻³ | 27.5 |
| ALS | | | | | | |
| Neurologically | 368 | 366 | 2 | 0.5% | | |
| normal | | | | | | |

To provide some insight into how an intermediate-length Ataxin2 polyQ repeat could enhance pathogenesis, we analyzed Ataxin-2 protein levels in lymphoblastoid cells from ALS cases harboring intermediate-length polyQ expansions, ALS cases with normal range repeat lengths, and controls. These studies showed that whereas the steady-state levels of Ataxin-2 were comparable, cycloheximide treatment, which blocks new protein synthesis, revealed an increase in stability of Ataxin-2 in cells with intermediate-length polyQ repeats (FIG. 4c,d). Thus, intermediate-length repeats increase Ataxin-2 stability, which could result in an increase in the effective concentration of Ataxin-2. This may further promote TDP-43 pathology beyond the interactions of Ataxin-2 harboring normal repeat lengths.

For a subset of the ALS cases (n=65; screened negative for mutations in SOD1, TARDBP, and FUS/TLS), extensive clinical details have been assessed. We interrogated the clinical characteristics in this cohort and compared the ALS cases with (n=8) and without (n=57) intermediate-length Ataxin-2 repeats. Strikingly, this analysis revealed that the age of onset was significantly advanced in ALS patients with intermediate-length Ataxin-2 repeats (Table 2, mean age 47.8 yrs vs. 59.4 yrs in ALS without intermediate-length Ataxin-2 repeats, p=0.03). ALS cases with intermediate-length Ataxin-2 repeats also had a higher frequency of family history of ALS (25.0% vs. 12.3%) and longer disease duration (mean of 64 months vs. 44.2 months) compared to ALS cases without intermediate-length Ataxin-2 repeats, although these differences were not statistically significant (Table 2). Because we observed an elevated frequency of family history among the ALS cases with intermediate-length Ataxin-2 repeats, we then examined an additional cohort of exclusively familial ALS cases (n=23 unrelated individuals; screened negative for mutations in SOD1, TARDBP, and FUS/TLS) and defined the Ataxin-2 polyQ repeat length in these individuals. In this cohort, the proportion of cases with intermediate-length Ataxin-2 repeats was even higher (Table 1, p=1.7×10-3, odds ratio 27.5); 3 of 23 (13%) probands with familial ALS harbored one allele with an intermediate-length Ataxin-2 repeat (mean repeat length 27, range 24-33). Taken together, these data suggest a causative link between intermediate length polyQ expansions in Ataxin-2 and ALS.

TABLE 2
Clinical features of ALS patients with intermediate-length
Ataxin-2 polyQ repeat expansions compared to ALS patients
with normal Ataxin-2 alleles. Repeat expansions showed significant
association with decreased age of onset.

| | 22-23 Repeats | 24-33 Repeats | |
|---|---|---|---|
| | ALS (n = 57) | ALS (n = 8) | P-value |
| Age of Onset (mean yrs, | 59.4 | 47.8 | 0.03 |
| 95% CI) | (55.8-63.0) | (34.8-60.9) | |
| Age of Death (mean yrs, | 62.3 | 56.6 | 0.19 |
| 95% CI) | (59.3-65.3) | (46.2-67.0) | |
| Duration (mean # of months, | 44.2 | 64.0 | 0.25 |
| 95% CI) | (31.1-57.3) | (10.3-117.7) | |
| ALS-FRS (mean, 95% CI) | 31.3 | 30.0 | 0.74 |
| | (28.4-34.2) | (21.8-38.2) | |
| % Male | 64.9% | 50.0% | 0.45 |
| % family history of ALS | 12.3% | 25.0% | 0.30 |

CI = confidence interval;
FRS = functional rating score

### DISCUSSION

We present evidence for intermediate-length polyQ expansions in the Ataxin-2 gene as a contributing cause of ALS. This finding extends from a simple modifier screen in yeast for genes whose activity affects TDP-43 toxicity. Confirmation of these studies in the fly and human cells, followed by biochemical analysis in yeast and human cells revealed that Ataxin-2 and TDP-43 can associate in a complex, and that the interaction depends on RNA. Further, Ataxin-2 is abnormally localized in ALS patient motor neurons, and TDP-43 pathology characterizes SCA2. Whereas long polyQ expansions in Ataxin-2 are the cause of SCA2, our studies reveal that intermediate-length polyQ expansions of 24-33 are associated with ALS, with a frequency of 4.5% in cases unselected for family history and ˜13% in familial ALS. These findings indicate that intermediate-length polyQ expansions in Ataxin-2 may be the most common cause of ALS defined to date; more than twice as common as mutations in SOD1 (2% of ALS cases), and much more common than mutations in FUS/TLS and TARDBP (<0.5% of cases).

The physical and genetic interactions between TDP-43 and Ataxin-2 suggest a model whereby Ataxin-2 serves as a bridge, either directly or via RNA, to bring TDP-43 to sites of a toxic function. Consistent with this, deleting Pbp1 in yeast or Ataxin-2 in the fly mitigated TDP-43 toxicity. Mutating the RRMs of TDP-43, in addition to blocking the interaction with Ataxin-2, eliminated TDP-43 toxicity. These data implicate Ataxin-2 as an essential mediator, likely via protein-protein or protein-RNA interactions, of TDP-43 toxicity in the cytoplasm. We did not observe a native interaction between both endogenous TDP-43 and Ataxin-2 (M.P.H and A.D.G. unpublished observations), supporting the notion that the TDP-43/Ataxin-2 interaction is associated with pathogenesis, rather than reflecting the normal physiological state. Because of this and the findings that reduction of Ataxin-2 suppresses TDP-43 toxicity in yeast and flies, the Ataxin-2/TDP-43/RNA complex may define a critical new target for therapeutic intervention in disease.

PolyQ repeat lengths in Ataxin-2 exceeding 34 cause SCA2²⁵⁻²⁸. Perplexingly, if the expanded trinucleotide repeats that encode the polyQ tract are not comprised of pure CAG, but rather interrupted with CAA (also encoding glutamine), evidence suggests patients are more likely to present with levo-dopa responsive parkinsonism than classic spinocerebellar ataxia⁴¹⁻⁴⁵. Our studies now indicate that intermediate-length Ataxin-2 polyQ repeat expansions are potentially a genetic cause of ALS. These findings are consistent with a model in which the Ataxin-2 repeat expansion is dominant, as has been observed in all the SCAs and most polyQ diseases. How then do different alterations in a single gene, ATXN2, contribute to at least three distinct clinical presentations (SCA2, parkinsonism, and ALS)? Long polyQ repeats in Ataxin-2 have been shown to increase aggregation³⁹ whereas smaller repeat expansions do not. While not wishing to be bound by theory, aggregated Ataxin-2 could have toxic gain-of-function properties that cerebellar Purkinje neurons are particularly sensitive to, resulting in SCA2. Intermediate polyQ expansions are not predicted to be aggregation-prone³⁹ and therefore could function to bring TDP-43 to its toxic location in the cytoplasm, where it is perhaps more deleterious to motor neurons, resulting in ALS. Additionally, polyQ expansions of different lengths could alter in different ways the protein-protein and/or protein-RNA complexes with which Ataxin-2 normally associates. For example, polyQ expansions in another Ataxin protein, Ataxin-1, which cause spinocerebellar ataxia 1 (SCA1), shift the balance of Ataxin-1 from one complex containing Capicua to another complex containing RBM17⁴⁶, resulting in both gain- and loss-of-function interactions mediated by the same mutation.

More globally, intermediate-length Ataxin-2 polyQ expansions could also strain the cellular proteostasis machinery akin to other disease situations^{47,48} in a way that favors cytoplasmic accumulation and aggregation of TDP-43. Given our findings of a critical role in ALS, this might be just the tip of an iceberg for Ataxin-2, which could contribute to the pathogenesis of many other diseases in which TDP-43 has a role. Determining which subset of ALS cases, as well as other TDP-43 proteinopathies, involve Ataxin-2 may facilitate further stratifying disease cases, which will ultimately aid the development of effective therapeutic approaches.

The identification of a novel and potentially common ALS disease gene from a simple yeast screen underscores the extraordinary power of yeast as a model system for gaining additional insight into human disease pathogenesis. There is no cure for ALS and currently the only treatment is riluzole, which slows disease progression by only 3 months⁴⁹. The identification of pathological interactions between Ataxin-2 and TDP-43, together with the strong genetic association of Ataxin-2 intermediate-length polyQ expansions and ALS, will empower the development of new therapies for this devastating human disease.

### Example II

### Diagnostic Assays for Detecting Increased Risk of Developing ALS with Early Onset

The information herein above can be applied clinically to patients for diagnosing an increased susceptibility for developing early onset ALS, and for therapeutic intervention. Diagnostic compositions, including microarrays, and methods can be designed to identify the polyQ expansions in the ataxin-2 gene described herein in nucleic acids from a patient to assess susceptibility for developing ALS. This can occur after a patient arrives in the clinic; the patient has blood drawn, and using the diagnostic methods described herein, a clinician can detect intermediate length polyQ expansions in the ataxin-2 gene. The nucleic acid obtained from the patient sample, which can optionally be amplified prior to assessment, will be used to diagnose a patient with an increased or decreased susceptibility for developing ALS. Kits for performing the diagnostic method of the invention are also provided herein. Such kits comprise a microarray comprising at least one probe or primer provided herein in and the necessary reagents for assessing the patient samples as described above. As discussed at length in Example I, the presence of intermediate length CAG repeats (˜24-33) in ATXN-2 is significantly associated with ALS, particularly early onset ALS. As mentioned previously a variety of assays are available for detection of polyglutamine expansions. Any of these assays may be utilized to detect the presence of intermediate length repeats for the diagnosis of an increased risk for the development of ALS.

The isolation of ataxin-2 genes comprising intermediate length polyQ repeats will serve to identify those that possess an altered risk for developing ALS. The information provided herein allows for therapeutic intervention at earlier times in disease progression that previously possible.

### Example III

### Screening Assays for the Identification of Agents which Modulate Pathological TDP-43-Ataxin-2 Complex Formation

Certain aspects of the present disclosure provide methods of screening for a candidate drug (agent or compound) or a genetic factor that modulates TDP-43-Ataxin-2 interactions and associated pathology. Various types of candidate drugs may be screened by the methods described herein, including nucleic acids, polypeptides, small molecule compounds, and peptidomimetics. In some cases, genetic agents can be screened by contacting the yeast cell with a nucleic acid construct coding for a gene. For example, one may screen cDNA libraries expressing a variety of genes, to identify other genes that modulate TDP-43-Ataxin-2 interactions. For example, the identified drugs may modulate TDP-43-ataxin-2 complex formation, subcellular localization and/or neuronal cell morphology or viability. Accordingly, irrespective of the exact mechanism of action, drugs identified by the screening methods described herein are expected to provide therapeutic benefit to patients suffering from ALS.

Screening methods described herein use may employ the yeast cells described in Example I. Candidate drugs can be screened from large libraries of synthetic or natural compounds. One example is an FDA approved library of compounds that can be used by humans. In addition, compound libraries are commercially available from a number of companies including but not limited to Maybridge Chemical Co. (Trevillet, Cornwall, UK), Comgenex (Princeton, N.J.), Microsource (New Milford, Conn.), Aldrich (Milwaukee, Wis.), AKos Consulting and Solutions GmbH (Basel, Switzerland), Ambinter (Paris, France), Asinex (Moscow, Russia), Aurora (Graz, Austria), BioFocus DPI, Switzerland, Bionet (Camelford, UK), ChemBridge, (San Diego, Calif.), ChemDiv, (San Diego, Calif.), Chemical Block Lt, (Moscow, Russia), ChemStar (Moscow, Russia), Exclusive Chemistry, Ltd (Obninsk, Russia), Enamine (Kiev, Ukraine), Evotec (Hamburg, Germany), Indofine (Hillsborough, N.J.), Interbioscreen (Moscow, Russia), Interchim (Montlucon, France), Life Chemicals, Inc. (Orange, Conn.), Microchemistry Ltd. (Moscow, Russia), Otava, (Toronto, ON), PharmEx Ltd. (Moscow, Russia), Princeton Biomolecular (Monmouth Junction, N.J.), Scientific Exchange (Center Ossipee, N.H.), Specs (Delft, Netherlands), TimTec (Newark, Del.), Toronto Research Corp. (North York ON), UkrOrgSynthesis (Kiev, Ukraine), Vitas-M, (Moscow, Russia), Zelinsky Institute, (Moscow, Russia), and Bicoll (Shanghai, China). Combinatorial libraries are available and can be prepared. Libraries of natural compounds in the form of bacterial, fungal, plant and animal extracts are commercially available or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

For example, the yeast cells in Example 1 can be incubated in the presence and absence of a test compound the effect of the compound on TDP-43/ataxin-2 complex formation and associated cellular toxicity assessed. Agents so identified could then be tested in whole animal models of ALS to assess in vivo efficacy.

Agents identified using the screening assays described herein are also encompassed by the present invention

### REFERENCES

1. Cleveland, D. W. & Rothstein, J. D. From Charcot to Lou Gehrig: deciphering selective motor neuron death in ALS. Nat Rev Neurosci 2, 806-19 (2001).
2. Valentine, J. S. & Hart, P. J. Misfolded CuZnSOD and amyotrophic lateral sclerosis. Proc Natl Acad Sci USA100, 3617-22 (2003).
3. Ou, S. H., Wu, F., Harrich, D., Garcia-Martinez, L. F. & Gaynor, R. B. Cloning and characterization of a novel cellular protein, TDP-43, that binds to human immunodeficiency virus type 1 TAR DNA sequence motifs. J Virol 69, 3584-96 (1995).
4. Buratti, E. et al. Nuclear factor TDP-43 and SR proteins promote in vitro and in vivo CFTR exon 9 skipping. Embo J 20, 1774-84 (2001).
5. Strong, M. J. et al. TDP43 is a human low molecular weight neurofilament (hNFL) mRNA-binding protein. Mol Cell Neurosci 35, 320-7 (2007).
6. Buratti, E. & Baralle, F. E. Multiple roles of TDP-43 in gene expression, splicing regulation, and human disease. Front Biosci 13, 867-78 (2008).
7. Neumann, M. et al. Ubiquitinated TDP-43 in frontotemporal lobar degeneration and amyotrophic lateral sclerosis. Science 314, 130-3 (2006).
8. Pesiridis, G. S., Lee, V. M. & Trojanowski, J. Q. Mutations in TDP-43 link glycine-rich domain functions to amyotrophic lateral sclerosis. Hum Mol Genet. 18, R156-62 (2009).
9. Lagier-Tourenne, C. & Cleveland, D. W. Rethinking ALS: the FUS about TDP-43. Cell 136, 1001-4 (2009).
10. Kwong, L. K., Uryu, K., Trojanowski, J. Q. & Lee, V. M. TDP-43 proteinopathies: neurodegenerative protein misfolding diseases without amyloidosis. Neurosignals 16, 41-51 (2008).
11. Winton, M. J. et al. Disturbance of nuclear and cytoplasmic TAR DNA-binding protein (TDP-43) induces disease-like redistribution, sequestration, and aggregate formation. J Biol Chem 283, 13302-9 (2008).
12. Bilen, J. & Bonini, N. M. Drosophila as a model for human neurodegenerative disease. Annu. Rev. Genet. 39, 153-171 (2005).
13. Caldwell, G. A. & Caldwell, K. A. Traversing a wormhole to combat Parkinson's disease. Dis Model Mech 1, 32-6 (2008).
14. Gitler, A. D. Beer and Bread to Brains and Beyond: Can Yeast Cells Teach Us about Neurodegenerative Disease? Neurosignals 16, 52-62 (2008).
15. Krobitsch, S. & Lindquist, S. Aggregation of huntingtin in yeast varies with the length of the polyglutamine expansion and the expression of chaperone proteins. Proc Natl Acad Sci USA 97, 1589-94 (2000).
16. Outeiro, T. F. & Lindquist, S. Yeast cells provide insight into alpha-synuclein biology and pathobiology. Science 302, 1772-5 (2003).
17. Cooper, A. A. et al. Alpha-synuclein blocks ER-Golgi traffic and Rab1 rescues neuron loss in Parkinson's models. Science 313, 324-8 (2006).
18. Gitler, A. D. et al. The Parkinson's disease protein alpha-synuclein disrupts cellular Rab homeostasis. Proc Natl Acad Sci USA 105, 145-50 (2008).
19. Gitler, A. D. et al. Alpha-synuclein is part of a diverse and highly conserved interaction network that includes PARKS and manganese toxicity. Nat Genet 41, 308-15 (2009).
20. Outeiro, T. F. et al. Sirtuin 2 Inhibitors Rescue {alpha}-Synuclein-Mediated Toxicity in Models of Parkinson's Disease. Science (2007).
21. Steffan, J. S. et al. Histone deacetylase inhibitors arrest polyglutamine-dependent neurodegeneration in. Drosophila. Nature 413, 739-43 (2001).
22. Johnson, B. S., McCaffery, J. M., Lindquist, S. & Gitler, A. D. A yeast TDP-43 proteinopathy model: Exploring the molecular determinants of TDP-43 aggregation and cellular toxicity. Proc Natl Acad Sci USA. 105, 6439-44 (2008).
23. Johnson, B. S. et al. TDP-43 is intrinsically aggregation-prone, and amyotrophic lateral sclerosis-linked mutations accelerate aggregation and increase toxicity. J Biol Chem 284, 20329-39 (2009).
24. Orr, H. T. & Zoghbi, H. Y. Trinucleotide repeat disorders. Annu Rev Neurosci 30, 575-621 (2007).
25. Imbert, G. et al. Cloning of the gene for spinocerebellar ataxia 2 reveals a locus with high sensitivity to expanded CAG/glutamine repeats. Nat Genet 14, 285-91 (1996).
26. Lorenzetti, D., Bohlega, S. & Zoghbi, H. Y. The expansion of the CAG repeat in ataxin-2 is a frequent cause of autosomal dominant spinocerebellar ataxia. Neurology 49, 1009-13 (1997).
27. Pulst, S. M. et al. Moderate expansion of a normally biallelic trinucleotide repeat in spinocerebellar ataxia type 2. Nat Genet 14, 269-76 (1996).
28. Sanpei, K. et al. Identification of the spinocerebellar ataxia type 2 gene using a direct identification of repeat expansion and cloning technique, DIRECT. Nat Genet 14, 277-84 (1996).
29. Infante, J. et al. Spinocerebellar ataxia type 2 with Levodopa-responsive parkinsonism culminating in motor neuron disease. Mov Disord 19, 848-52 (2004).
30. Nanetti, L. et al. Rare association of motor neuron disease and spinocerebellar ataxia type 2 (SCA2): a new case and review of the literature. J Neurol (2009).
31. Mangus, D. A., Amrani, N. & Jacobson, A. Pbp1p, a factor interacting with Saccharomyces cerevisiae poly(A)-binding protein, regulates polyadenylation. Mol Cell Biol 18, 7383-96 (1998).
32. Buchan, J. R., Muhlrad, D. & Parker, R. P bodies promote stress granule assembly in Saccharomyces cerevisiae. J Cell Biol 183, 441-55 (2008).
33. Auluck, P. K., Chan, H. Y., Trojanowski, J. Q., Lee, V. M. & Bonini, N. M. Chaperone suppression of alpha-synuclein toxicity in a Drosophila model for Parkinson's disease. Science 295, 865-8 (2002).
34. Warrick, J. M. et al. Suppression of polyglutamine-mediated neurodegeneration in Drosophila by the molecular chaperone HSF'70. Nat Genet 23, 425-8 (1999).
35. Buratti, E. & Baralle, F. E. Characterization and functional implications of the RNA binding properties of nuclear factor TDP-43, a novel splicing regulator of CFTR exon 9. J Biol Chem 276, 36337-43 (2001).
36. Lastres-Becker, I., Rub, U. & Auburger, G. Spinocerebellar ataxia 2 (SCA2). Cerebellum 7, 115-24 (2008).
37. Tharun, S. Roles of eukaryotic Lsm proteins in the regulation of mRNA function. Int Rev Cell Mol Biol 272, 149-89 (2009).
38. Oubridge, C., Ito, N., Evans, P. R., Teo, C. H. & Nagai, K. Crystal structure at 1.92 A resolution of the RNA-binding domain of the U1A spliceosomal protein complexed with an RNA hairpin. Nature 372, 432-8 (1994).
39. Huynh, D. P., Yang, H. T., Vakharia, H., Nguyen, D. & Pulst, S. M. Expansion of the polyQ repeat in ataxia-2 alters its Golgi localization, disrupts the Golgi complex and causes cell death. Hum Mol Genet 12, 1485-96 (2003).
40. Geser, F. et al. Evidence of multisystem disorder in whole-brain map of pathological TDP-43 in amyotrophic lateral sclerosis. Arch Neurol 65, 636-41 (2008).

While the invention has been described in detail and with reference to specific examples thereof, it will be apparent to one skilled in the art that various changes and modifications can be made therein without departing from the spirit and scope thereof.

What is claimed is:

1. A method for predicting an increased risk of an individual for developing amyotrophic lateral sclerosis (ALS) disease, said method comprising: obtaining a nucleic acid sample encoding ataxin-2 from said individual and determining whether or not said ataxin-2 comprises intermediate length polyglutamine expansions relative to wild-type ataxin-2 encoding nucleic acids, wherein the presence of an ataxin-2 intermediate-length polyglutamine expansion within said ataxin-2 relative to wild type ataxin-2 is indicative of an increased risk of ALS.

2. The method of claim 1, wherein said method predicts an increased risk of early onset ALS.

3. The method of claim 1, wherein said nucleic acid is obtained from a blood, tissue or skin sample.

4. The method of claim 1, wherein said polyglutamine expansions are detected using an ATXN2 specific probe or primer.

5. The method of claim 1, comprising the steps of
a) amplifying genomic ataxin-2 containing nucleic acid obtained from said individual using oligonucleotide primers of SEQ ID NO: 1 and SEQ ID NO: 2 thereby obtaining an amplified PCR product; and
b) determining CAG repeat length in said product.

6. The method of claim 1, wherein said polyglutamine repeat length is between 24 and 33 glutamine residues.

7. The method of claim 6, wherein said polyglutamine repeat length is between 25 and 28 glutamine residues.

8. A diagnostic kit for performing the method of claim 1, comprising reagents suitable for isolation of DNA, and reagents suitable for detection of ataxin-2 encoding nucleic acid comprising said CAG repeats.

9. A diagnostic kit for performing the method of claim 5, said kit comprising SEQ ID NO: 1 and SEQ ID NO: 2, reagents suitable for isolation of genomic DNA from said individual, reagents suitable for performing PCR and wild type ataxin-2 and ALS associated ataxin-2 encoding nucleic acids for use as negative and positive controls.

10. A method for identifying agents which inhibit TDP-43-ataxin-2 complex formation, said ataxin-2 containing intermediate length polyQ expansions, comprising:
a) providing a cell which expresses TDP-43 and said ataxin-2, said expression being associated with increased cellular toxicity and cytoplasmic aggregate formation;
b) contacting said cell with an effective amount of an agent; and
c) measuring cellular toxicity and/or aggregate formation in the presence of said agent relative to a non-treated control cell, wherein a decrease in cellular toxicity identifies an agent which reduces TDP-43-ataxin-2 mediated cellular toxicity and cytoplasmic aggregate formation.

11. The method of claim 10, wherein said cell is a Saccharomyces cerevisiae cell.

12. The method of claim 10, wherein said agent to be identified is obtained from a compound library comprising natural or synthetic compounds.

13. An agent identified via the method of claim 12.

14. A composition comprising the agent of claim 13 present in a biologically acceptable carrier.
